(19) United States
(12) Patent Application Publication
(10) Pub. No.: US 2025/0053790 A1
(43) Pub. Date: Feb. 13, 2025

(54) CAUSAL FRAMEWORK FOR REAL-WORLD EVIDENCE GENERATION WITH LANGUAGE MODELS

(71) Applicant: Microsoft Technology Licensing, LLC, Redmond, WA (US)

(72) Inventors: Javier GONZÁLEZ HERNANDEZ, Cambridge (GB); Hoifung POON, Bellevue, WA (US); Cliff WONG, Seattle, WA (US); Zelalem Hailu GERO, Redmond, WA (US); Jaspreet Kaur BAGGA, Seattle, WA (US); Emre Mehmet KICIMAN, Seattle, WA (US); Aditya Vithal NORI, Cambridge (GB); Tristan Josef NAUMANN, Cambridge, MA (US); Risa UENO, Cambridge (GB); Eduard ORAVKIN, London (GB)

(21) Appl. No.: 18/533,166

(22) Filed: Dec. 7, 2023

Related U.S. Application Data
(60) Provisional application No. 63/518,555, filed on Aug. 9, 2023.

(51) Int. Cl. G06N 3/0455; G06F 30/27; G06N 3/0475; G06N 3/09; G16H 10/60

(52) U.S. Cl. CPC G06N 3/0455; G06F 30/27; G06N 3/0475; G06N 3/09; G16H 10/60

(57) ABSTRACT

Example solutions for real-world evidence generation using artificial intelligence models and performing trial simulations include: training a large language model (LLM) to receive medical documents that include medical text associated with a patient output predicted values for medical attributes of the patient based on the medical text; performing attribute extraction from structured medical documents, including extracting values for a first plurality of attributes associated with the plurality of patients; performing attribute extraction from a plurality of unstructured medical documents of the plurality of patients using the LLM, including extracting predicted values for a second plurality of attributes associated with the plurality of patients; and performing a survival model simulation that computes estimations of hazard ratio (HR) between cases and controls using real-world data of the plurality of patients extracted in the first attribute extraction and second attribute extraction.

### CROSS-REFERENCE TO RELATED APPLICATION

This application claims the benefit of U.S. Provisional Patent Application No. 63/518,555 filed on Aug. 9, 2023, which is hereby incorporated by reference herein in its entirety.

### BACKGROUND

Rapid digitization of real-world data offers an unprecedented opportunity for optimizing healthcare delivery and accelerating biomedical discovery. In practice, however, such data is most abundantly available in unstructured forms, such as clinical notes in electronic medical records (EMRs) and/or electronic health records (EHRs). Moreover, this data is often plagued by confounders (e.g., variables that are related to both an independent variable and a dependent variable in a given study).

### SUMMARY

The disclosed examples are described in detail below with reference to the accompanying drawing figures listed below. The following summary is provided to illustrate some examples disclosed herein. The following is not meant, however, to limit all examples to any particular configuration or sequence of operations.

Example solutions for performing trial simulations include: training a large language model (LLM), the LLM being configured to: receive, as at least one input, a medical document that includes medical text associated with a patient; and generate, as at least one output in response to the at least one input, one or more predicted values for one or more medical attributes of the patient based on the medical text included in the medical document; performing first attribute extraction from a plurality of structured medical documents of a plurality of patients, thereby extracting values for a first plurality of attributes associated with the plurality of patients; performing second attribute extraction from a plurality of unstructured medical documents of the plurality of patients using the LLM, thereby extracting predicted values for a second plurality of attributes associated with the plurality of patients; and performing a survival model simulation that computes estimations of hazard ratio (HR) between cases and controls using real-world data of the plurality of patients extracted in the first attribute extraction and second attribute extraction

### BRIEF DESCRIPTION OF THE DRAWINGS

The disclosed examples are described in detail below with reference to the accompanying drawing figures listed below:

FIG. 1 illustrates an example trial simulation system that provides a large language model (LLM) framework that enables the end-to-end inference and validation of the causal effect of biomedical interventions by using language models to structure raw electronic health records (EHRs);

FIG. 2 illustrates additional details and aspects of data flow for components of a trial simulation system such as shown in FIG. 1 in preparation for performing an example trial simulation;

FIG. 3A provides a graphical description of the notes collected in the medical journey of a lung cancer patient;

FIG. 3B illustrates details regarding an example selection process for simulations performed by a system such as shown in FIG. 1;

FIG. 4 illustrates an example architecture that uses a data structuring pipeline to extract these attributes from the EHRs;

FIG. 5 is a table summarizing the approach taken to extract each group of attributes as well as the ground truth data used to validate the extraction;

FIG. 6 provides a summary of key metrics of performance of the models, including the area under the precision-recall curve, the area under the receiver operating characteristic curve, and test prediction accuracy for multi-task classification problems;

FIG. 7 shows the available unique patients in the dataset that can be extracted for lung cancer event, ECOG and three medications (Doxetacel, Pembolizumab and Cisplatin);

FIG. 8 is a table illustrating results from a group of simulations performed with the system of FIG. 1 and FIG. 2;

FIG. 9 contains summary statistics for variables;

FIG. 10 illustrates other tests that were performed;

FIG. 11 is a flowchart illustrating exemplary operations that may be performed by a system such as shown in FIG. 1 for performing trial simulations using patient medical data extracted from structured data sources and unstructured/semi-structured data sources; and

FIG. 12 is a block diagram of an example computing device (e.g., a computer storage device) for implementing aspects disclosed herein.

Corresponding reference characters indicate corresponding parts throughout the drawings. Any of the drawings may be combined into a single example or embodiment.

### DETAILED DESCRIPTION

Real-world evidence (RWE) refers to the use of real-world data (RWD) collected from sources other than randomized controlled trials (RCTs) to produce clinical evidence about the benefits or risks of medical treatments. The Food and Drug Administration (FDA) Real World Evidence Program, the NICE report (National Institute for Health and Care Excellence), and other regulatory bodies recognize the potential of RWE to complement RCTs in a broad range of patients, settings, and outcomes. A recent example of the success of RWE is use of Palbociclib (PAL) (a cyclin-dependent kinase 4/6 inhibitor) to treat breast cancer in males. The rarity of breast cancer in men limited the feasibility of a RCT in this population. RWE was used to approve the treatment for male individuals post-market without the need of a RCT.

RWE is cheaper to collect and can be used to answer hypotheses beyond those posed in RCTs, typically designed with the aim of running a differential analysis between two drugs. As an example, the patient journey recorded in EHRs may span a time-frame beyond the duration of a trial, making it possible to infer long term patient outcomes otherwise inaccessible because of the constrained follow-up duration of the trial. But RWE also poses several challenges, such as data quality, heterogeneity, and bias, which impact their regulatory validity and slows down its adoption as a standard tool for assisted biomedical decision making. RWE has difficulties in replicating existing evidence from randomized trials on several occasions. In one example, RWE was used to show that surgery was superior to radiotherapy for oropharynx Cancer, but this claim was later refuted with an RCT. There have been other examples where observational medical studies have been refuted by RCTs.

Generating RWE from medical records typically relies upon assumptions that are hard to test in practice. The most common one is the identification of all possible sources of confounding bias (e.g., factors that have an influence in the response while simultaneously making patients in the treatment and controls groups to be different before any treatment is provided). Under this scenario, disentangling whether the differences between the groups are due to the effects of the therapy or due to these factors becomes the cornerstone of any RWE analysis. In RCTs, this can be achieved by randomization, moving the challenge from running an unbiased statistical analysis to defining an efficient patient recruiting process. However, another issue with RWE studies is the amount of patient information that is presented in the form of unstructured text. This source of information has been traditionally ignored by most studies, leading to the mentioned biases and lack of reproducibility. Although registry data like demographics, vital status, and so forth, are usually fairly ready for analysis, structured data is usually incomplete and may not always be available for all patients at all times. Such data also lacks an overall clinical context.

In artificial intelligence (AI), general purpose language models trained in a large corpus of text like BERT, LLAMA, and GPT-4 have proliferated. In the biomedical domain, specific language models trained in biomedical datasets, like PubMedBERT or BioGPT have also been developed in the last few years showing an increase in performance in several biomedical tasks.

In examples, a unifying framework for distilling real-world evidence from population-level observational data is provided. A trial simulation system leverages LLMs to predict patient attributes and structure EMR data from the RWE at scale, employs advanced probabilistic modeling for data denoising and imputation, and incorporates state-of-the-art causal inference techniques to combat common confounders.

During a model training stage, the trial simulation system trains an attribute extraction LLM to identify values for particular attributes from various types of semi-structured and unstructured healthcare documents. The training data may, for example, be a set of documents labeled with one or more attributes and their associated values. In examples, the LLM is trained as a classification model to generate a predicted value for a given attribute (e.g., where the domain of the attribute is a non-contiguous space) or as a regression model (e.g., where the domain of the attribute is a contiguous space). As such, when provided a particular document and a particular attribute, the LLM is trained to identify, from that document, a predicted value for that particular attribute, as well as perhaps a confidence score that identifies how likely that predicted value is to be correct.

During a preparation stage, the trial simulation system generates an initial RWE matrix for a set of patients. The RWE matrix includes a row for each individual patient, as well as columns that each represent a particular health attribute. As such, each cell of a given row represents a value for an attribute for that particular patient. Some values for these attributes can be easily extracted from structured data sources, such as healthcare databases (e.g., where some attributes are predefined fields and may be populated with values for each patient). Other attributes may be more difficult to identify as they may only appear in unstructured or semi-structured documents, such as clinical notes. For these attributes, the trial simulation system performs attribute extraction on the semi-structured or unstructured data sources using the LLM to generate values for those particular attributes for each patient.

As such, the semi-structured and unstructured data sources are used with the LLM to populate some or most of the attributes that are missing from the structured data sources. However, some values for particular attributes may be completely missing for particular patients, or may be present but may be corrupt or bad (e.g., as a low confidence value from the LLM output). As such, the trial simulation system may also use a latent variable model on the corrupt patient data to generate predictions for those corrupt or missing attributes. These predictions are then entered into the RWE matrix to further refine that matrix.

During a simulation stage, the trial simulation system uses the RWE matrix to perform a trial simulation (e.g., a survival analysis, time-to-event analysis, or the like). In examples, the system uses patient selection criteria to identify a population of patients from the full RWE matrix to generate a “trial RWE matrix” (e.g., a subset of rows of the full RWE matrix). This selection criteria can be based on one or more of the attributes from the RWE matrix (e.g., attributes identified from the structured data sources or attributes extracted, derived, or otherwise identified from the semi-structured or unstructured data sources). This trial RWE matrix is then used by the system to perform the trial simulation. In examples, the system uses the trial RWE matrix to perform a Cox Proportional-Hazards (CoxPH) model on the selected population of patients based on the attributes identified in the trial RWE matrix. In some examples, the system may also use inverse propensity score weighting (IPSW) along with CoxPH (e.g., CoxPH-IPSW, where confounders are incorporated with weights). The simulation results may be compared to known trials to value the overall success of the simulation.

Using clinical trial specification as generic representation, the trial simulation system provides a turn-key solution to generate and reason with clinical hypotheses using observational data. In extensive experiments and analysis on a large-scale real-world dataset with over one million cancer patients from a large U.S. healthcare network, the trial simulation system is shown to produce high-quality structuring of real-world data and often generates comparable results as marquee cancer trials. In addition to facilitating in-silicon trial design and optimization, the trial simulation system may be used to empower synthetic control, pragmatic trials, and post-market surveillance, as well as support fine-grained “patient-like-me” reasoning in precision diagnosis and treatment.

While aspects of the disclosure are described with reference to LLMs, other aspects of the disclosure are operable with multimodal models, visual models, and the like.

The various examples are described in detail with reference to the accompanying drawings. Wherever preferable, the same reference number is used throughout the drawings to refer to the same or like parts. References made throughout this disclosure relating to specific examples and implementations are provided solely for illustrative purposes but, unless indicated to the contrary, are not meant to limit all examples.

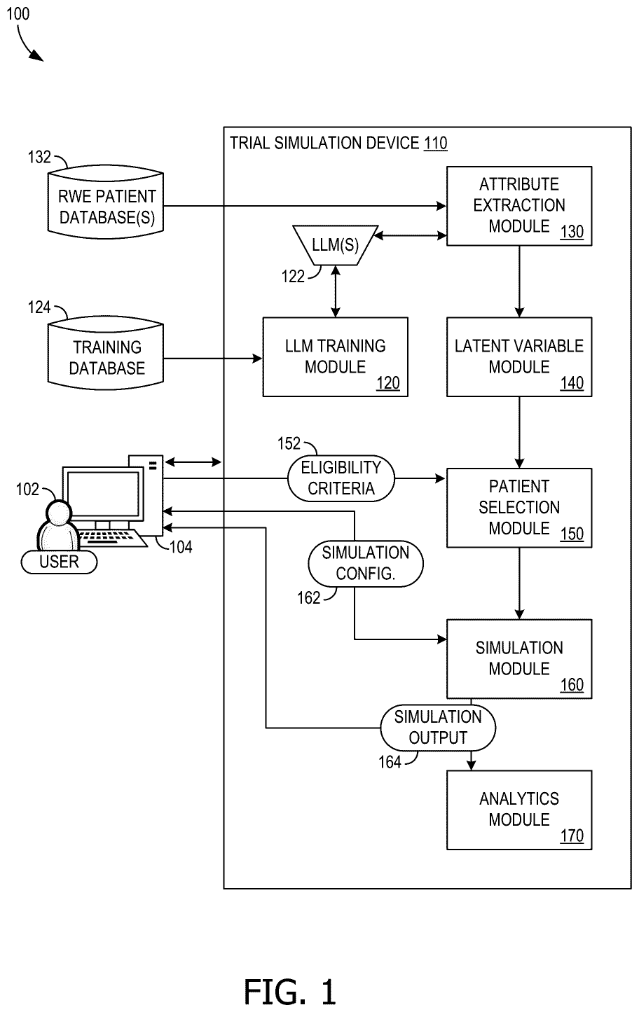

FIG. 1 illustrates an example trial simulation system 100 that provides an LLM-based framework that enables the end-to-end inference and validation of the causal effect of biomedical interventions by using language models to structure raw EHRs. Examples provided herein present the main elements of the system 100 and show a proof-of-concept in the Oncology domain. These examples focus on the simulation of previously published advanced non-small cell lung cancer (aNSCLC) trials using structured and unstructured EHRs data from a large integrated delivery network (IDN) comprising healthcare systems in five western US states. This disease is used in these examples because it has a large prevalence of data, but generalizations to other use-cases can be achieved straightforwardly. Also, although the analysis presented here is mainly retrospective, the same principles and methods can be used to generate unknown evidence beyond previous RCTs.

The trial simulation device 110 facilitates the simulation of clinical trials with reduced human data curation. More specifically, in examples, a trial simulation device 110 of the system 100 includes six main components. A LLM training module 120 is configured to train an LLM 122 that will be used in trial simulations. An attribute extraction module 130 performs data structuring by using the LLM 122 to automatically extract attribute values from semi-structured and unstructured data sources for patients (e.g., medical notes of patients from RWE patient database(s) 132), thereby generating a unified, structured dataset. A latent variable module 140 performs data imputation on the dataset to refine the “imperfect” patient attribute data generated by the attribute extraction module 130 (e.g., deriving missing or corrupt attribute values for patients).

After the dataset has been refined, the trial simulation device 110 performs a trial simulation using some subset of that dataset. More specifically, in examples, a patient selection module 150 selects multiple patients for a target trial simulation (e.g., matching patients with RCT by applying eligibility criteria 152 based on logical statements over the dataset). A simulation module 160 allows a user 102 (e.g., via a user computing device 104) to configure a trial simulation (e.g., via configuring eligibility criteria 152, simulation configuration 162) and perform a simulation of the target trial using the eligible patients and associated data. In some examples, the simulation module 160 performs a Causal CoxPH model simulation that computes estimations of the hazard ratio (HR) between cases and controls (e.g., using inverse propensity weighting to simulate the target trial). An analytics module 170 analyzes simulation output 164 of the trial simulation to identify whether or not the simulation can be trusted (e.g., generating test diagnostics used to evaluate the quality of the simulation output 164).

In examples, the LLMs 122, when used as biomedical text structuring tool, and in combination with causal methods, can generate RWE with the potential of providing regulatory validity able to complement RCTs. The reliance on RWD to provide regulatory validity can be integrated on a spectrum of cases, ranging between traditional RCTs (e.g., where no RWD is needed) and non-randomized non-interventions studies (e.g., where the reliance of on RWD is significant). LLMs 122 are used in the integration of RWD in this spectrum. In-silicon trial design and optimization, synthetic control, pragmatic trials, post-market surveillance, as well as support fine-grained patient-like-me reasoning in precision diagnosis and treatment are just some examples of what this system 100 can achieve.

In prior approaches, a bag-of-words representation of free-text is used to discover interpretable confounders (e.g., medical terms) that help to reduce the gap between the estimated HR and those observed in previous prostate and lung cancers.

In contrast, here, the LLMs 122 are used to process the EHRs with minimal data curation. More specifically, the LLMs 122 extract high quality data structuring for specific variables of interest which may or may not be reflected in any single word of the text. As such, the trial simulation system 100 is able to simulate RCTs using raw electronic heath records as input. This system 100 has been conceived with the idea of facilitating the generation and validation of biomedical hypothesis at scale with minimal human data curation and maximum transparency. As such, this system 100 helps to increase the use of RWE to inform clinical practice by serving as a complement to expand RCTs by increasing the number of biomedical questions that can be generated and tested.

During a model training phase, the trial simulation device 110 trains one or more LLMs 122 to be used for trial simulation. More specifically, in examples, the LLM training module 120 trains at least one LLM 122 as a classification model and/or as a regression model to generate a predicted values for one or more particular attributes of a patient from a given medical document of that patient (e.g., EHR, EMR, or the like). During the training phase, the user 102 identifies a set of training documents (e.g., in training database 124) to use during training of the LLM 122. These training documents can be, for example, clinical notes, pathology reports, progress notes, imaging reports, or the like. Each of these training documents are labeled with values for one or more of the particular attributes present in the document (e.g., for supervised learning). This set of training documents can include various types of documents, any of which may sometimes include values for some particular attributes. Given a large variety of document types, styles, and ways of expressly or implicitly identifying values for particular attributes within those documents, the LLM 122 can thus be trained to predict values for those particular attributes from a new input document.

The user 102 thus identifies the various attributes and the attribute types (e.g., possible values for that attribute) that can be predicted by the LLM 122 based on the various labels attached to the training documents. Attributes can include categorical attributes (e.g., where the attribute has one or more values from a given set of possible values), continuous attributes (e.g., where the attribute can take any value within a range), binary attributes (e.g., where the attribute is one of two possible categories or classes), text attributes (e.g., a collection of words or characters), or image attributes (e.g., pixel data representing an image). Further, the LLM 122 may include a scoring function that can be used as a summary measure for the prediction (e.g., a probability that the predicted value is correct).

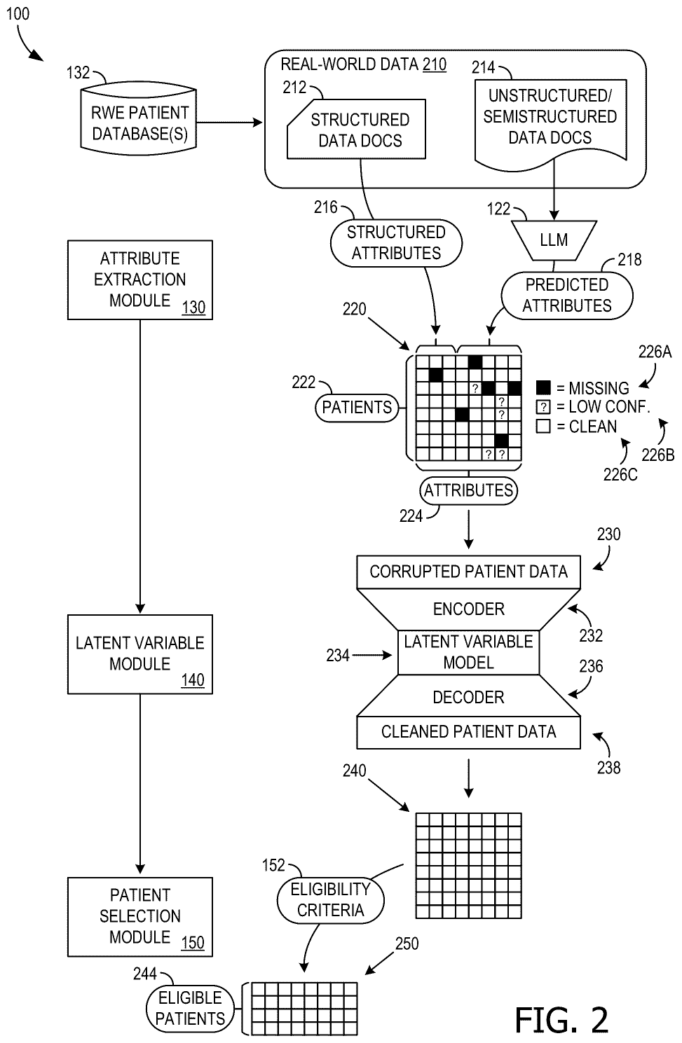

FIG. 2 illustrates additional details and aspects of data flow for components of the trial simulation system 100 shown in FIG. 1 in preparation for performing an example trial simulation. In this example, it is presumed that the LLM 122 has previously been trained to predict values for various medical attributes (e.g., by the LLM training module 120 as described in FIG. 1).

In examples, the system 100 uses a medical database (e.g., RWE patient database 132) as a data source for medical records (e.g., EHRs, EMRs) of patients, represented here as real-world data 210. This real-world data 210 includes structured data documents (docs) 212 and unstructured/semi-structured data docs 214, and it is presumed that any particular document of the real-world data 210 can be identified to a particular patient. Structured data docs 212 represent data sources that are formatted such that values for particular attributes can be directly extracted (e.g., formatted as field/value pairs, or the like, such as with relational database tables, NoSQL data sources such as JSON-formatted data sources, object-oriented data sources, XML documents, or the like). Example attributes that may be derived from structured data docs 212 can include patient age, gender, smoking, alcohol use, diagnosis codes, biomarkers, medications history, lab test history, sequencing results, or other demographic data or medical data of the patient. Given the structured nature of these structured data docs 212 and the structured attributes 216 contained there, the attribute extraction module 130 can directly read values for particular attributes from these docs 212. The attributes and their associated values extracted from structured data docs 212 are shown here as structured attributes 216.

Unstructured/semi-structured data docs 214, in the example, represent data sources that are not structured data docs 212. An unstructured document is a document that does not inherently provide a data structure for any attributes. A semi-structured document is a document that may provide data structure for some data, but the attributes and values of interest are contained in the document (either expressly or implicitly) in an unstructured form (e.g., not necessarily within a field/value pair provided by the document). Example unstructured/semi-structured data docs 214 can include pathology reports, imaging reports, progress notes, encounter notes, surgery notes, or the like (e.g., where data is provided in free-form text, perhaps input by medical professionals during past treatment of the patient). Such docs 214 can also include scanned images or PDFs of such documents that are scanned to identify text within those images (e.g., via an optical character recognition (OCR) system or the like). These docs 214 can include or otherwise be used to infer values for various attributes, such as biomarkers, medications, histology, pathological stage data, site data, tumor progression data, ECOG scores, and the like. Since the data in such docs 214 is not provided in a structured format, the attribute extraction module 130 uses the LLM 122 to predict values for a given patient using the docs 214 of that patient. The attributes and their associated values extracted from unstructured/semi-structured data docs 214, as identified by the LLMs 122, are shown here as predicted attributes 218.

The attribute extraction module 130 uses the structured attribute 216 and predicted attributes 218 extracted from the real-world data 210 to build an RWE matrix 220. The RWE matrix 220 includes rows of patients 222 (e.g., one row for each individual patient) and columns of attributes 224 (e.g., one column for each particular attribute). Each row of the RWE matrix 220 is populated with the values for each attribute extracted for that particular patient 222 (e.g., the structured attributes 216 and predicted attributes 218 extracted from the particular docs 212, 214 associated with that patient). As such, each cell of the matrix 220 represents a value for the attribute 224 of a particular patient 222.

In the example, not all cells are initially similarly populated for all patients 222. Structured attributes 216 are considered “clean” cells 226C, as there is a high confidence that the values extracted from structured data docs 212 are accurate. Some predicted attributes 218 from the unstructured/semi-structured data docs 214 may or may not be as reliable. As such, the attribute extraction module 130 may mark cells of the matrix 220 as clean cells 226C when the value for that particular predicted attribute 218 for that patient is over a confidence threshold, but otherwise may mark cells of the matrix 220 as “low confidence” cells 226B when the value for that particular predicted attribute 218 is below the confidence threshold (represented in FIG. 2 as “?” cells). Further, some values for attributes 224 may not be identified for a given patient 222 at all (e.g., from either the structured data docs 212 for that patient 222 or the unstructured/semi-structured data docs 214 for that patient 222). As such, these cells are identified as “missing” 226A (represented in FIG. 2 as black cells).

Upon construction of the RWE matrix 220, the attribute extraction module 130 has created an initial version of this matrix 220. However, and as shown in FIG. 2, there are several missing values for certain attributes 224 of certain patients 222 in the matrix 220. As such, in the example, the latent variable module 140 is configured to refine the initial RWE matrix 220 using a latent variable model 234. More specifically, the latent variable module 140 uses the RWE matrix 220 as corrupted patient data 230 and uses the latent variable model 234 to infer values for the missing or low-confidence cells 226A, 226B based on the observed data for that patient 222 (e.g., treating the cells 226A, 226B as unobserved or latent variables and the clean cells 226C as observed data). In this example, the latent variable module 140 uses an encoder 232 to prepare the data 230 for use with the model 234 (e.g., into a numerical format) and a decoder 236 to decode the resultant data back into the format of the matrix 220 (represented as cleaned patient data 238 in FIG. 2). The latent variable module 140 thus populates inferred values for the missing/low-confidence data (e.g., the cleaned patient data 238) into the matrix 220, resulting in a refined RWE matrix 240. This refined RWE matrix 240 is shown in FIG. 2 as having all clean cells 226C, but it should be understood that, in some scenarios, some missing or low-confidence cells 226A, 226B may still be present.

The patient selection module 150, in the example, uses the eligibility criteria 152 to identify eligible patients for a trial simulation. More specifically, the patient selection module 150 selects a subset of the patients 222 from the refined RWE matrix 240, and based on the eligibility criteria 152, to create a trial RWE matrix 250 that will be used for the trial simulation. This selection process can include, for example, inspecting each patient 222 (e.g., each row) in the refined RWE matrix 240, comparing particular attributes 224 of that patient 222 against the eligibility criteria 152 to determine whether or not that patient 222 satisfies the eligibility criteria 152. For patients 222 that satisfy the eligibility criteria 152, the data for that row of the patient 222 is copied into the trial RWE matrix 250, thus making that patient one of the eligible patients 244 in the trial RWE matrix 250. Patients 222 that do not satisfy the criteria 152 are excluded from the trial RWE matrix 250.

As such, the simulation module 160 (shown in FIG. 1) uses the trial RWE matrix 250 as inputs to the trial simulation, thereby identifying the population of patients that will be used for the simulation (e.g., the eligible patients 244), as well as input data for each of those patients (e.g., the attributes 224 of the matrix 250).

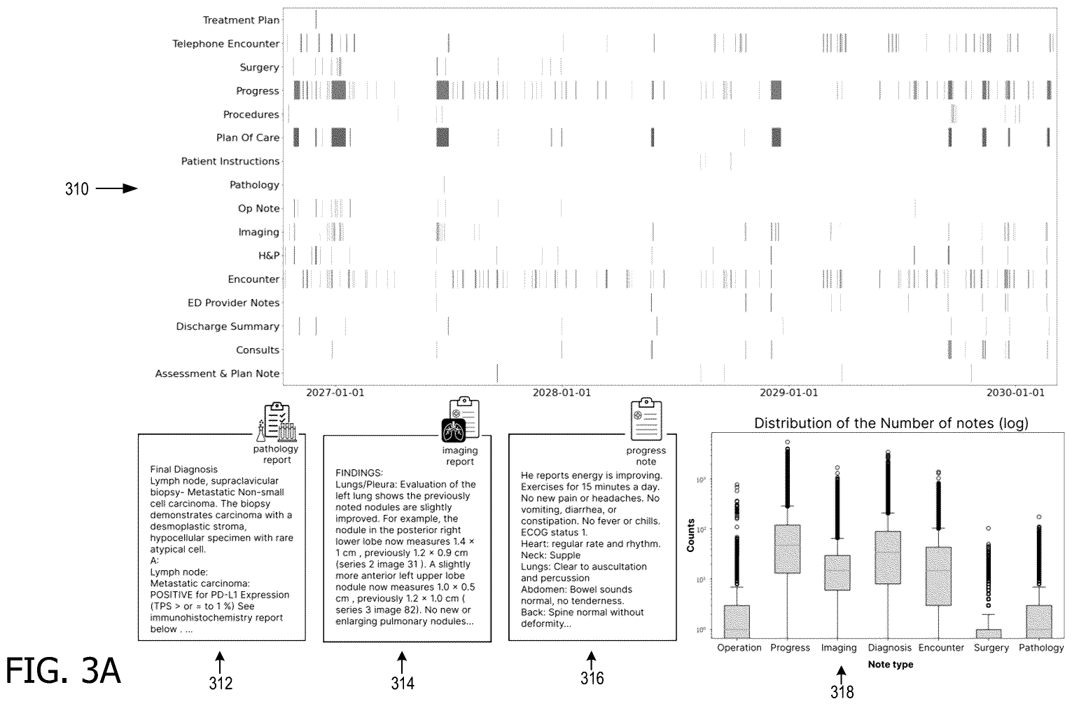

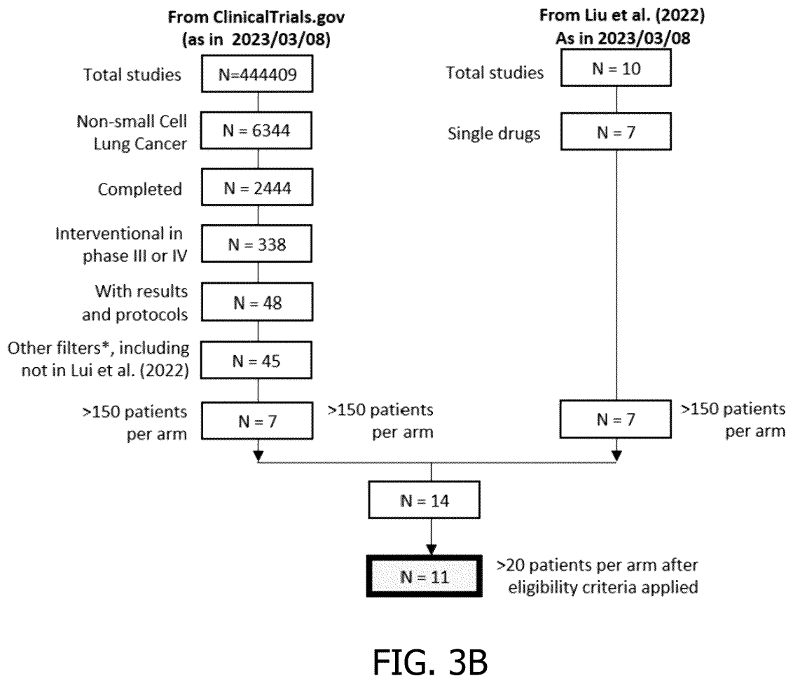

FIG. 3A provides a graphical description of the notes collected in the medical journey of a Lung Cancer patient. In examples, diagnosis codes may be generated for billing purposes, and thus may be a part of structured data docs 212 shown in FIG. 2. Free-text notes, on the other hand, are a wealthy source of information that does provide clinical context. The data shown in FIG. 3A provide examples of unstructured/semi-structured data docs 214 shown in FIG. 2. RWE may be generated at scale to enhance the comprehensiveness of the new biomedical evidence, reduce publication bias, and avoid P-hacking. More specifically, FIG. 3A illustrates the scale and complexity of the data collected during the medical journey of an example patient (time scale has been simulated). At graph 310, each bar represents a collected note. At box 312, an example pathology report covers the characteristics of a tissue specimen. At box 314, an example imaging report provides an accurate interpretation of images in a format that will prompt appropriate care for the patient. The report relates the findings about the patient's current clinical symptoms to the results of other investigative tests and procedures. At box 316, an example progress note is part of a medical record that keeps the ongoing record of the patient's illness and treatment. All reports in FIG. 3A have been generated for illustrative purposes and do not represent any specific patient. Graph 318 shows an example distribution of the most abundant notes of each type in the database 132.

FIG. 3B illustrates details regarding an example selection process for simulations performed by the system 100 (e.g., eligibility criteria 152). In examples, the RWE patient database 132, has a total of about three million patients with one million of those being Cancer patients. Each patient is represented by an extensive collection of EHRs of different types. To show the ability of the system 100 to produce valid biomedical evidence using these EHRs as raw input, 11 advanced Non-Small Lung Cancer (aNSCLC) randomized trials were simulated using the system 100. These example simulations focus on this disease due to its large prevalence in the database. An exhaustive trial selection process was performed to choose the trials following a combination of two sources: trials described in “Evaluating Eligibility Criteria of Oncology Trials Using Real-World Data and AI,” (Liu et al., 2021); and trials that satisfy certain characteristics in ClinicalTrials.gov. To facilitate the statistical significance of the results, the selection includes only trials in which at least 150 patients in the medical database had been exposed to the drugs of in both arms of the trials before the data was cleaned. Under these criteria, 14 trials were selected. However, only 11 had more than 20 patients in both arms after filtering the patients by eligibility criteria: FLAURA, CHECKMATE017, CHECKMATE057, KEYNOTE010, OAK, KEYNOTE024, STELLA, NCT001307285 (no code name available), NCT02604342 (no code name available), EMPHASIS and PROFILE014. The remaining 4 trials (LUXLUNG8, KEYNOTE033, LUXLUNG6, PROFILE1014) were excluded from the analysis.

In this example simulation, a total of 29, 020 aNSCLC patients had received the treatment in at least one of the arms in any of the 14 selected trials. Patients were included in the analysis if they were diagnosed with lung Cancer (as per the international classification of diseases and had pathology consistent with NSCLC) with stage IIIA, IIIB, IIIC, IVA or IVB. For each trial, the eligibility criteria was encoded based on which data were available in the medical database. Each eligibility criterion is implemented as a logical statement, which is applied to the structured dataset of records. The eligibility of the trials is satisfied for the closest value to the start date of the therapy. Not all criteria are applicable in all the trials. Patients were also excluded if they had more than two years between diagnosis and treatment and if they had inconsistent start and diagnosis dates.

The analysis focused on the overall survival of the patients, a metric commonly reported in RCTs. The analysis used the published results from the selected trials to judge whether the process of structuring, filtering and modeling the EHRs provides evidence that is consistent with the known effects captured by the RCTs. The analysis used the hazard ratio between the treatment and control groups as the key quantity to evaluate the correctness of the evidence provided using the dataset. The HR is also a commonly reported metric in Lung Cancer trials. It is assumed that the HR published in the documentation of each trial captures the ground truth effect. In this example, a trial is considered successfully simulated when the HR obtained with the medical database statistically overlaps the result published in the original trial.

In more than 90% of the cases that were analyzed, strong statistical evidence was found of the equivalence between the simulation are statistically equivalent. In cases where the evidence was weaker, it was found that the differences have a plausible explanation. To characterize these differences, validation tests are provided that are able to identify when the results from trials and simulation are expected to be comparable.

Longitudinal data was obtained from patient records by means of the system 100, which uses both structured data (e.g., the structured data docs 212) and unstructured data (e.g., the unstructured/semi-structured data docs 214). This provides a holistic, comprehensive view of the patient's journey and speeds up the process of structuring patient data (e.g., into the form of the matrices 220, 240, 250).

In naive approaches, it is expected for a trained curator to expend about one hour to extract data for one patient. However, with the data extraction pipeline and methods described herein, the system 100 can achieve the same work in just a few minutes of computing time.

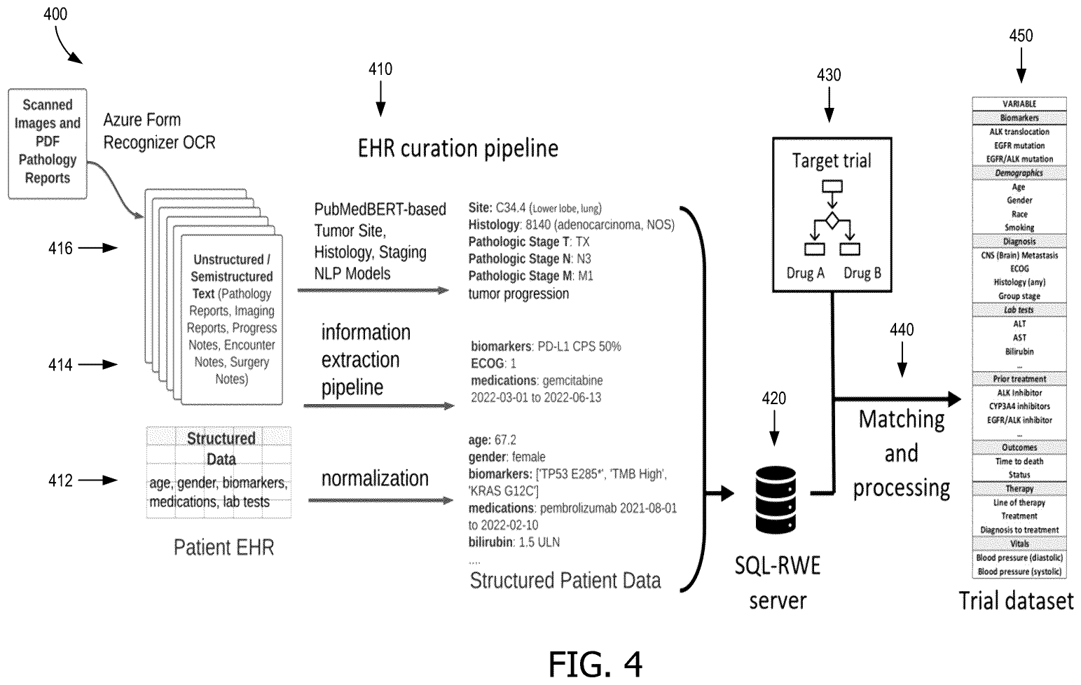

FIG. 4 illustrates an example architecture 400 that uses a data structuring pipeline 410 to extract these attributes from the EHRs. The pipeline 410 includes three main components: a data pipeline for structured data 412; an information extraction pipeline (IEP) 414 based on a sequence of natural language processing tasks; and a language model structuring pipeline (LMP) 416. FIG. 4 shows the pipeline used to structure the EHRs to obtain a database 420 that is suitable to simulate a trial 430. In examples, the architecture 400 may be similar to aspects of the system 100 shown in FIG. 1 and FIG. 2. For example, the patient EHR shown in FIG. 4 may be the real-world data 210 of FIG. 2, where the data pipeline for structured data 412 is similar to the structured data docs 212, and where the information extraction pipeline 414 and LMP 416 is similar to the unstructured/semi-structured data docs 214 and LLM 122 of FIG. 2. In this example, a target trial 430 represents the trial simulation being performed and a trial dataset 450 represents the patient data being used for that trial after a matching process 440 is performed (e.g., similar to the eligibility criteria 152 used to create the trial RWE matrix 250 of FIG. 2).

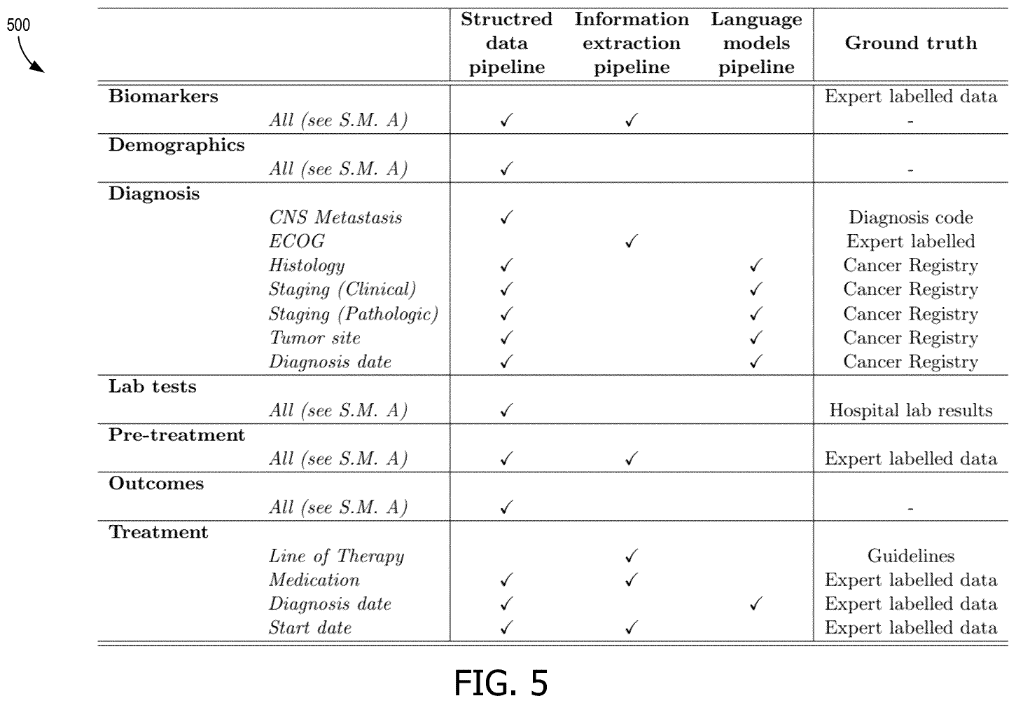

FIG. 5 is a table 500 summarizing the approach taken to extract each group of attributes (attribute-level details given when needed) as well as the ground truth data used to validate the extraction. The choice of the method depends on the availability and quality of structured data, the difficulty of the task, and the importance of the data for our analysis. When the same information from several sources was available, the value extracted using the method with higher confidence was selected when necessary. For structured data, the data was normalized from different sources to the same ontology or unit. No validations were performed since the extract attributes are extracted from the records.

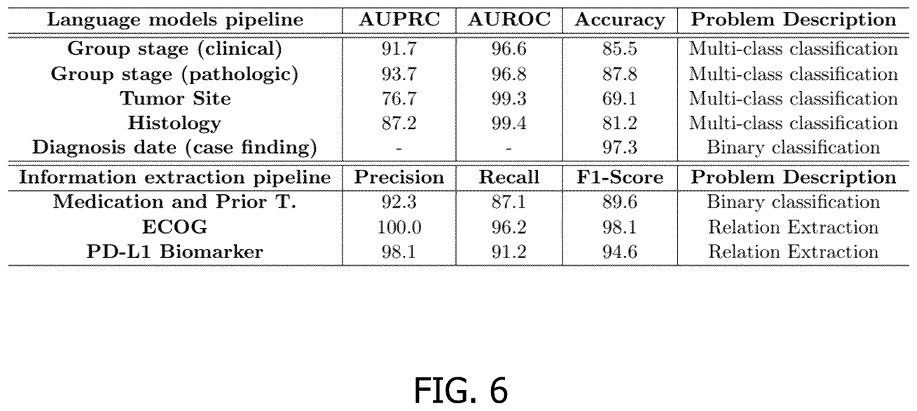

Structuring EHRs in the form of free text is an information extraction problem where the task is to extract the values for all relevant patient attributes from a given set of clinical documents. This task is specifically challenging in this setting, where the input document includes all the notes for a patient and variables of interest can take hundreds of unique values (e.g., tumor site has 310 classes). In examples, the LMP 416 (e.g., the LLM 122) uses fine-tuned language models based on PubMedBERT. This pipeline was built using medical database EHRs so it was also applicable in this setting. It is a combination of three deep learning techniques: a transformer-based, domain-specific foundation model to generate good sentence-level encoding; a recurrent neural network to propagate information across sentences; and hierarchical attention network to summarize information across multiple documents. This approach was used to process staging (clinical and pathological), tumor site, histology, and diagnosis date, as shown in the top of FIG. 6. Here, FIG. 6 provides a summary of key metrics of performance of the models, including the area under the precision-recall curve (AUPRC), the area under the receiver operating characteristic curve (AUROC), and test prediction accuracy for multi-task classification problems. For extraction of the tumor diagnosis date, F1-scores are reported.

The information extraction pipeline 414 is a combination of natural language processing techniques that were used to process ECOG score, medications, and PD-L1 biomarkers. The system 100, in the example, uses customized spaCy sentence segmentation and NLTK for tokenization. For extraction, the system 100 uses domain-specific rules to identify relevant entities, potential relations and determine if they are positive assertions. The bottom section of FIG. 6 shows results for the performance of structuring the medication, ECOG score, and PD-L1 biomarker.

A gold standard test is configured to evaluate ECOG by randomly selecting 565 cancer patients who had active cancer after a given date and with the selection of one progress note per patient within nine months after that date. All the progress notes in the test set were dated after the notes used to develop the domain-specific rules. A domain expert manually annotated those progress notes and labeled 79 notes with an explicit performance score. The average word length per note in this test set is 1015.2 word tokens. To construct the ground truth test set for PD-L1 biomarkers, 135 cancer patients with PD-L1 mentions in their notes were randomly sampled, taking into account various surface forms of PD-L1 (PDL1, PDL-1, PDL 1, etc.). For each patient, one progress note or pathology report was randomly sampled that contained a PD-L1 mention and is dated after the notes used to develop the domain-specific rules. A domain expert manually extracted PD-L1 biomarker expression levels and the score type (combined positive score, tumor proportion score) for all notes in the test set. This resulted in 171 unique patient-expression level-score type relations. The extraction was evaluated by correctly matching both the expression level and score type.

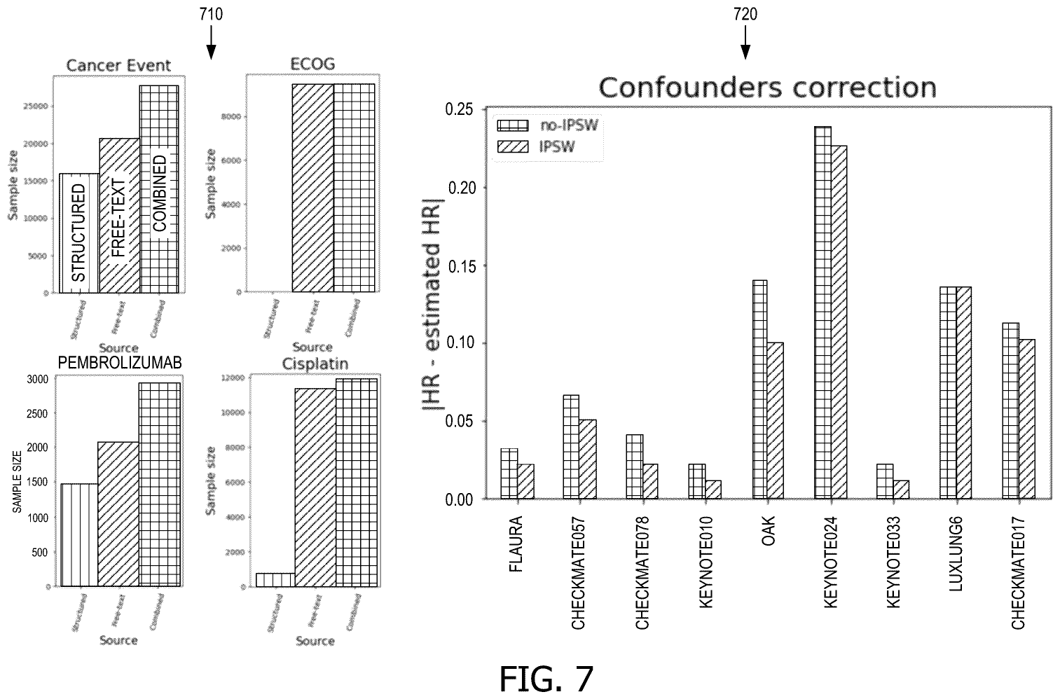

Combining structured and free-text ERHs into a unique extraction pipeline increases both speed and scope. Beyond having a way to rapidly extract new data, key attributes like ECOG and certain biomarkers may not be available in structured form. Also, by combining structured and structured data, the number of patients in the studies can be augmented. FIG. 7 shows the available unique patients in the dataset that can be extracted for (Lung) Cancer event, ECOG and two medications (Pembolizumab and Cisplatin) at graphs 710. This data is compared with available records in these variables by only using the structured data, only the free-text data, and the union of both. Using the free-text expands both the patient attributes that are accessible (e.g., like ECOG that is only accessible via free-text) and the number of patients. Graph 720 illustrates the effect of correcting the simulators by confounders in all of the trials. Graph 720 illustrates the absolute difference between the HR in the original trial and the estimation with and without IPSW. The estimation of the HR benefits remains the same in all the trials when confounders are added to the models.

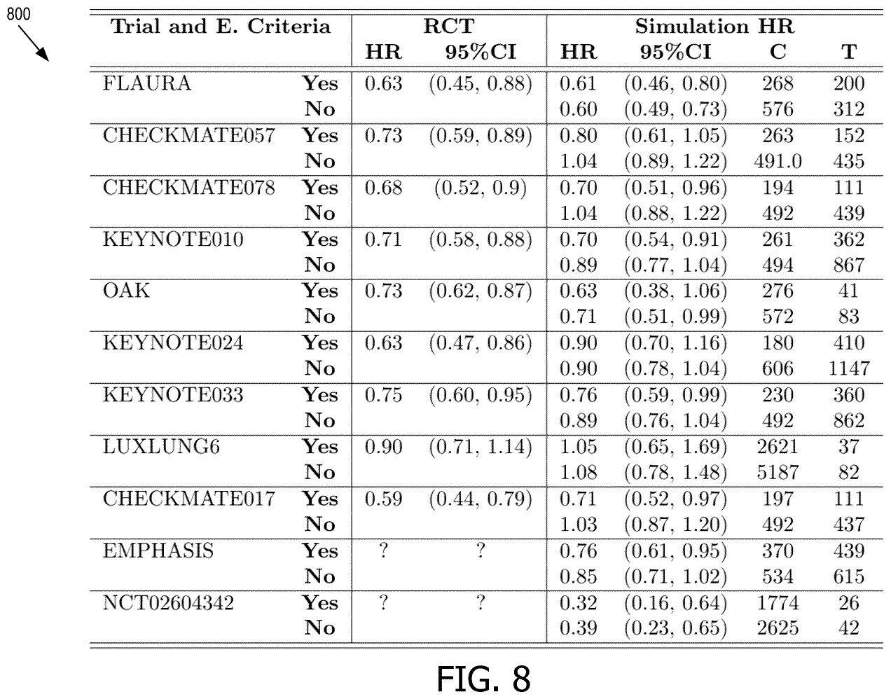

In examples, the system 100 was used to simulate a total of 11 aNSCLC completed clinical trials. The goal is to evaluate if the evidence produced by the system 100 is consistent with already validated biomedical evidence. The hazard ratio (HR) was used for the overall survival between the treatment and control as the key metric to evaluate the success of each simulation. Several pre-processing steps and filters are applied to the structured data to help ensure that the RCT and the simulations are as comparable as possible. This includes a step for missing data imputation and other data cleaning steps. The HR was computed for the treatment after applying all the eligibility criteria of the trials and when the full cohort based on the drugs only. In all the trials, the same data processing pipeline and the same modelling approaches were used, and Cox proportional hazards (CoxPH) model (Cox, 1972) was used in all the analysis. To account for biases due the lack of randomization in the data, a CoxPH model was used, where confounders are incorporated via inverse propensity score weighting (IPSW) with weights computed using logistic. To marginalize out the effect of other drugs taken by the patient, their presence was accounted for, and their effects marginalized in the final outcome model. All the trials were simulated with and without applying all their eligibility criteria. The results of the 11 simulated trials are summarized in FIG. 8.

FIG. 8 is a table 800 illustrating results from a group of simulations performed with the system 100 of FIG. 1 and FIG. 2. In this example, data and results from simulations of 11 selected single-drug trials are illustrated. In these example trials, CoxPH model with inverse propensity re-weighting was used. Each pair of rows in the table 800 contains the results for one trial. Rows labelled with “No” are results with datasets of patients that were filtered only by line of therapy and the treatment and control drugs of the trial. Rows in the table labelled with “Yes” also exclude patients filtered using all the eligibility criteria. The HR and its 95% confidence interval are shown for the original RCTs and for the simulations that use the medical database. The sample size of the treatment (T) and control (C) groups are also included. Note that the HR from the RCTs are only comparable to the simulation results in which patients are filtered by eligibility criteria (EC). From the total of 11 trials, two did not report the HR due the lack of statistically significant results (e.g., not enough patients). In the remaining nine trials, the simulations and the published trial are statistically equivalent in nine cases (e.g., >90% of the time). However, the evidence in the KEYNOTE024 trial is weaker.

In this example, the simulations and trials are consistent regardless of the value of the HR. Trials like FLAURA (HR-RCT: 0.63, n=556) or CHECKMATE057 (HR-RCT: 0.73, n=582), had HR<1 and were correctly simulated. The simulated HR for FLAURA was 0.61 (n=468) and for CHECKMATE057 0.80 (n=415). On the other hand, LUXLUNG6 trial (HR-RCT: 0.9, n=364) had a non-statistically equal-to-one HR. The simulations capture this effect with a value of HR=1.05 (n=2658). This is an important result. It highlights the correct alignment of the information provided by the structuring EMRs using language models with existing comparative evidence of lung cancer treatments. This occurs in cases where differences between groups are significant and also when they are not.

The simulation results are compared with and without applying the eligibility criteria (e.g., using line of therapy only). The differences in the estimated HRs with and without using all criteria vary from trial to trial. In KEYNOTE024, the variation is minimal (HR-No-Criteria: 0.90, and n=2753; HR-with-Criteria: 0.90, n=580), whereas in the CHECKAMATE057 the differences are larger (HR-no-Criteria: 1.04, n=926; HR-with-Criteria: 0.80, n=415). This provides evidence that for some trials the eligibility criteria can be relaxed while maintaining a similar level of significance in the results.

The effect of correcting the simulations using IPSW has a different effect depending on the trial. Graph 720 of FIG. 7 shows the differences for the absolute differences between the RCT HRs and the estimated HRs with and without the confounders correction in the Cox-PH model (when all the eligibility criteria are applied). Correcting for confounders moves the estimation of the HR towards the ground truth values in eight of nine cases with strong corrections in the CHECKMATE057 and OAK 6 trials. In LUXLUNG06 the correction does not have any effect in the result. This result shows that confounding correction helps to reduce bias in survival models built with EMRs.

Two of the selected trials, EMPHASIS and NCT02604342, did not report the HR. This is because these trials did not manage to collect enough patients and had to be discontinued. This is a well-known problem in clinical trials where a not-insignificant proportion of trials fail solely due to the inability to recruit eligible patients. Here, these are simulated in an exercise of forecasting what the HR may have been in case the trial had been successfully enrolled/completed. The simulation of the EMPHASIS trial resulted in an HR of 0.76 and a 95% confidence interval of [0.61, 0.95] (n=1009) and the NCT0260434 resulted in an HR of 0.32 and a 95% confidence interval of [0.16, 0.64] (n=1800). This exemplifies how RWE can be used as a way to collect early evidence of the result of a trial even before the first patients have been recruited.

The example simulations utilize causal assumptions that are not readily verifiable from data. Comparing with the HR in the RCT is the ultimate validation. In cases where a reference HR is not available, a set of validation tests is relied upon that aim to capture model behaviors that may indicate that some of the assumptions are broken. To illustrate these behaviors, EMPHASIS trial is used. Simulation for the EMPHASIS trial shows an HR=0.76 (95% CI=[0.16, 0.64], n=809). Because a reference HR is not available, other indicators of the goodness of this simulation are assessed that can be carried out without knowing the trial HR.

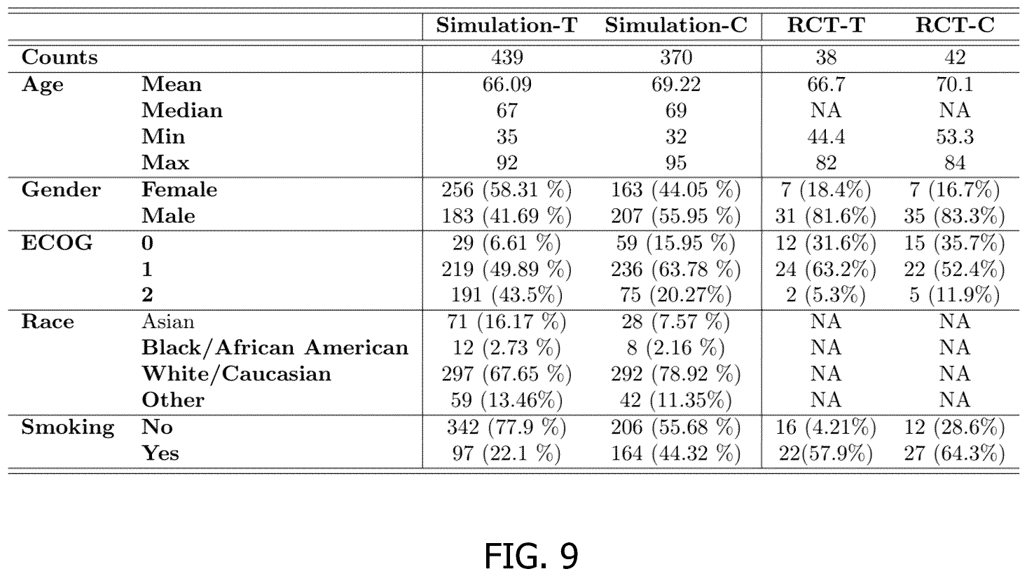

FIG. 9 contains summary statistics for the data in the original RCT publication and the simulation (Providence data) for the reported covariates in EMPHASIS (T=treatment, C=Control). NA stands for not available values. The two populations differ, which implies that the conclusions of the simulation cannot be extrapolated to the trial.

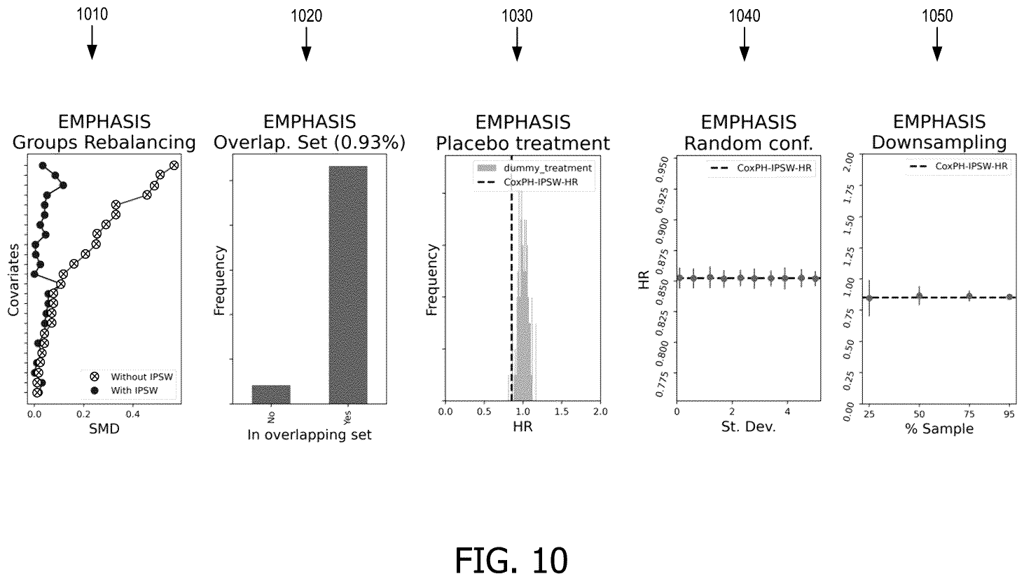

Other tests are also performed to investigate other sources of bias and variance in the simulation. FIG. 10 illustrates other tests that were performed. First, the balancing between treatment and control groups was tested before and after using the IPSW correction (e.g., shown as graph 1010). The standardized mean distance (SMD) was computed for all confounders in both cases. The differences are close to zero when the IPSW correction is applied and remain large when it is not. This indicates that the differences between the groups due to factors beyond the treatment are properly corrected. Second, we check the percentage of the data that with non-null probability could have been selected for either of the control or treatments. The larger is this set, the more suitable the dataset is for causal analysis. In EMPHASIS, 93% of the patient data in the RWD for this trial satisfies this property, as shown in graph 1020. Third, the strength of the signal is questioned. The assignment to the treatment group is randomly permuted 100 times and the HR is recomputed in each one of them. As expected, the signal vanishes when the patients are assigned randomly, as shown in graph 1030. Fourth, the robustness of the simulation is tested by adding a new zero-mean Gaussian confounder in the analysis, as shown in graph 1040. Several scenarios are run in which we systematically increase the standard deviation of the confounder in a grid of values between 0.1 and 5. 100 replicates are generated for each scenario and compute the 95% confidence interval for the replicates using the 5% and 95% percentiles. If the model is robust, adding this extra variable should not affect the estimation of the HRs and the variation should be minimal, which is what is observed in this experiment. Fifth, the HR is computed by randomly down-sampling the dataset and retaining only 95, 90, 75, 50, and 25% of the patients (100 repetition in each scenario). A well-behaved simulator should provide the same average HR in all cases, with an increase in variance when the sample size decreases. Again, this is what is observed in the case of CHECKMATE057, as shown in graph 1050. In summary, this trial is an exemplar of how a simulation is expected to behave.

In examples, this system 100 provides a new RWE-framework that demonstrates that language models (e.g., LLMs 122) in combination with causal tools can be used to reproduce evidence found previous single-drug aNSLC trials. In cases where the evidence is inconsistent, the system 100 has demonstrated that it is possible to explain the differences, which serves an unsupervised characterization of when the simulations can be trusted. This disclosure focuses on a proof-of-concept, the retrospective simulation of 14 aNSLC trials, but the framework presented here can be generalized to other scenarios where RWE can be exploited to improve decision making in the biomedical domain.

Accordingly, the system 100 can be used to improve clinical practice. Although the analysis presented in this paper are primarily retrospective, evidence about the comparative efficacy of drugs does not need to be limited to cases where a trial exists. With the increasing digitization of medical records, the system 100 can be used as a tool to extract insights from EHRs. Biomedical researchers can use the system 100 to generate knowledge at a speed that is order of magnitude faster than the current state of the art, in which information in free text is, if not directly ignored, structured manually or with limited digital support. The system 100 is able to incorporate the result of fully language model based structuring pipeline into causal inference approaches via a reweighed Cox-PH model. Also, the system 100 presents a novel combination of these approaches with an exhaustive set of diagnostics tools useful to add transparency when incorporating the system 100 in biomedical decision-making pipelines.

With the recent rapid development of artificial intelligence tools and language models like GPT-4, some of the components of the system 100 can rapidly evolve. While, in these examples, the data is structured by means of a combination of pubMedBERT-based information extraction pipeline, zero-shot data structuring via prompting with GPT-4 or similar models can also be performed. Further, the current system 100 may also perform the extraction of the eligibility criteria in an automated fashion. Automating this step will increase the scalability of the system 100, making it possible to simulate hundreds of trials.

In general, the distribution of the population in the RCT is unknown. This limits the capacity to evaluate how the population of patients in the RWD setting is comparable to the trial. In these examples, the system 100 used summary statistics of some key attributes, which is the only information that is available in published trials. Further, the causal modelling aspects of this system 100 have been kept intentionally simple to increase transparency. However, the CoxPH has the drawback that with many covariates the proportional hazard assumption may be violated. Other models can be used by the system 100. Among the potential extensions that are worth considering are Cox models that are time dependent or that model the confounding bias in the censoring mechanisms.

It is important to note that although the system 100 uses the HR to test the ability to simulate a trial because it is publicly available with RCT results, the HR does not have a causal interpretation even under patients randomization. The reason for this is that although the distributions of the populations are the same at the beginning of the study (due to randomization or confounding correction) this is not necessarily true as time progresses and the two sub-populations may become less balanced due to the loss of patients. An alternative here is to use the Causal HR, which addresses some of the potential issues.

An interesting consideration when computing the HR for a particular population is the uniformity of the response. The HR can be significant for the patients involved in a trial, but nothing guarantees the uniformity of the response across sub-populations. Fairness considerations may be considered to guarantee that no patient is left behind. This is an important problem that, although not mainstream in the clinical trials literature, has received some recent attention.

Combo trials are trials in which the treatment medication consist of a combination of drugs that are tested when they are administrated simultaneously. The simulation of combo trials is not shown in these examples, but the system 100 can be configured to address such simulations. What is observed in the dataset is that combo trials are more challenging to simulate than single drug trials. A hypothesis for this is the health state of the patients involved in the trials. Combo drugs are usually extreme treatments that are given to patients with very poor health, which may have an effect in the consistency and robustness of the results.

All the confounders used in these examples are based on previous analysis and factors that can simultaneously affect the assignment to the treatment and the response. Direct collaboration with subject matter experts can be used in this step. In other examples, however, the system 100 may implement systematic ways and statistically grounder ways of identifying sources of confounding will be key for the general adoption of ours and other RWE tools.

This system 100 is of interest to medical practitioners as well as researchers in the area. The workflows presented here serve as a reference for framing both problems and answers in RWE. The framework described herein can help drive the development and adoption of more complex approaches able to tackle a range of problems that go beyond the retrospective analysis of existing trials. Ultimately, the system 100 can help to increase transparency in the use of RWE, speeding up its adoption and improving patients' lives.

### Additional Methods

An example patient dataset includes electronic health records (EHR) from about 3.3 million patients. About 1 million of those patients are Cancer patients. From this dataset, patients were collected with advanced non-small-cell lung Cancer. For each patient clinical notes, history and physical notes, treatment plans, discharge summaries, etc., were extracted. In addition, semi-structured information about each patient was collected from the inpatient billing system as well as available information at the start of the treatment time. This includes cancer lab test results, staging, tumor description, date of diagnosis, date of death, date of the last follow-up for the selected patients. Demographics and other patient characteristics like age, gender, ethnicity are extracted from the structured patient data.

These example simulations used a systematic approach to select the trials to simulate. Considered for simulation were all single-drug trials with at least 150 patients in both arms before any data filtering applied. This set was augmented by conducting a search in ClinicalTrials.gov. Because this is an evolving dataset, the search was fixed with the following filters: Non-small Cell Lung Cancer trials (6344 studies), completed studies (2444 studies), that are interventional in phase III or IV (338 studies), with study protocols (48 studies) and that have two arms with different treatments with available reports and that were not already in the work by Liu et al. (2021) (45 studies). For consistency, trials with at least 150 patients in both arms were selected before any data processing. This leads to a total of nine extra eligible trials. Finally, another five trials proposed by a subject matter expert were considered, two of which were included due the same sample availability criterion (>150 patients per arm).

A cohort of patients per trial was built. A selection of all the patients that resemble as much as possible a population that could have been eligible for each trial while maximizing the number of patients per trial. It was infeasible to isolate patients with a single-drug treatment while maintaining a large enough sample. Patients were selected that received at least a dose of the target drug. To remove the effect of other potential drugs taken by the patient, a dictionary of drugs was extracted. A binary vector that captures the presence or absence of each drug in each patient is computed. This vector is then used in the Cox-PH model to remove the effect of drugs that are different to the target ones. The databases contain a total of one million patients, 29,020 of which took the control or target treatment in at least one of the analyzed trials. Patients were also selected according to the line of therapy of the trials. If the line of treatment was missing, this criteria was ignored and the patient added to the trial as in Liu et al. (2021).

Before selecting patients for the trials according to their eligibility criteria, other data cleaning filters were applied. These filters aim to simulate as much as possible the data collected for the original trials and remove noise and bias. Patients with inconsistent start diagnosis and death dates were removed as well as those individuals with more than two years between diagnosis and treatment. All treatment patients were removed who took the control drug during “trial” and the control patients that took the treatment drug. Any duplicated patients were removed with clear data inconsistencies. Patients for which the event (death or last visit) was recorded in a time beyond the duration of the trial were corrected. All of these patients were set to have a duration the same as the trial. If the patient died beyond the threshold, he/she is assumed to be alive and the data point censored. All the data processing was done with in python using pandas and it is part of the code library.

Testing the correctness of biomedical interventions, Cancer treatments in particular, it is helpful to be able to systematically test and diagnose the models that generate new RWE. To address this issue, a Python-based framework, the system 100, has been developed that is easy to use and extend.

Patient demographics refer to the date of birth, gender, race, ethnicity, vital status, last contact date, death date, and so forth. This information comes structured from the hospital's internal records. However, for the death date, we extract this information from both the hospital records and social security death records. If a patient's death information is not found, we extract the last contact date by using the latest date from all records for that patient.

In examples, for oncology attributes (e.g., site, histology, staging, diagnosis date), structured data from the cancer registry is combined with predictions from self-supervised PubMedBERT language model. The LLM inputs include pathology reports, progress notes, imaging reports, encounter notes, diagnosis code description, Op Notes, and Surgery notes. The model predicts ICD-O-3 site and histology codes, which are then mapped to OncoTree IDs for clinical trial matching. To predict diagnosis date, the case-finding model is used only on pathology reports.

In examples, biomarkers are obtained in structured form but is normalized to three fields: gene, variant, and variant type. Variant is normalized to HGVS nomenclature when possible. However, some biomarkers are only available in unstructured form, such as pathology reports that contain third-party laboratory test results. For PD-L1 IHC results, an information extraction pipeline is used that contains three steps: entity extraction, relation extraction, and intent detection. The entity extraction step extracts PD-L1 test names such as combined positive score (CPS), Tumor Proportion Score (TPS). Result values are also tested, such as negative, positive, high, low, a specific percentage value (e.g., 10%), or a range of percentage values (e.g., >50%). Relation extraction is then applied to determine if there is a relation between that PD-L1 test and the test result value. If so, the intent of that relation is classified as to whether or not this patient actually has that biomarker relation. Oftentimes, patient notes may contain biomarker entities, but the patient may not actually have that biomarker. The text may contain a description of the biomarker test, writing about a hypothetical situation, or negation. The date of the PD-L1 measurement mentioned in the note is not extracted but the note date is used for the PD-L1 measurement. In some examples, the system also extracts the date associated with each PD-L1 measurement.

In examples, information about patients' medication is extracted from both structured and unstructured medical records. Two sources of structured medication information are used-“Ordered-Meds” and “Administered-Meds”. Ordered-Meds contains all medications ordered for a patient which may or may not have been administered to the patient. Administered-Meds are all medications that are given to the patient at the hospital and are of high confidence. Using medication descriptions from the structured data, each medication is standardized to NCI (National Cancer Institute) Thesaurus concept ID. This standardization allows for a seamless use of drug synonyms, abbreviations, and collective names interchangeably. The medication extraction module complements the structured medication information with data extracted from free clinical notes since neither all patients have structured medication nor the once who have structured medication information have a complete medication history. Clinical notes contain more comprehensive and detailed patient drug information, but such information is typically buried in large unstructured format and is thus not easily accessible. In examples, the treatment extraction pipeline is used to extract medication information from free clinical texts and has the following modules:
Extract medications module—the first step in the pipeline, extracts medication mentions, these can be in short-forms, medication codes or any of the drug synonyms;
Extract attributes module—for each of the extracted medications, attributes from the span are identified, and the most important ones are dosage (e.g., amount of a medication used in each administration), frequency (e.g., how often each dose should be taken), mode (e.g., route for the medication), date (e.g., date of medication administration), and discontinuation/Substitution (e.g., whether the medication is still active or being discontinued/substituted);
Link Entities module—here, each of the extracted attributes are linked to their corresponding medication;
Determine Administration module—using all the information above, this module determines whether the medication mentioned in the clinical note is administered to the patient or not. This is a challenge since medications can be mentioned in a clinical note as a suggestion, in reference to past history, or in a hypothetical framing.
   Using the treatment extraction pipeline and the structured medication information, complete treatment information about a patient is identified. Each patient data will be enriched with details such as the list of all medications taken with dose/frequency/mode, date of each administration, when substitutions/discontinuations happened in the patient timeline.

Determining the Line of Therapy (LoT) helps to assess a patient's eligibility for a given clinical trial. However, there are no universally accepted set of criteria to enumerate LoT. To alleviate this, the system 100 adopts the guidelines suggested in the works of Saini and Twelves (2021); Meng et al. (2021) to determine line of therapy. The following is the final guideline we have followed to determine LoT in our cohort of patient. First, the L1 LoT is defined as the first SACT (systemic anti-cancer therapy) drug recorded after date of diagnosis. Second, if clinical progression of disease is documented, assign a new LOT to the next SACT. Third, if a SACT drug is discontinued and substituted by another drug of the same class, retain the same LoT. Fourth, if one or more new anti-cancer agents are added to an ongoing SACT, assign a new LoT. Fifth, if one or more anti-cancer agents are discontinued from an ongoing SACT, retain the same LoT for the remaining anti-cancer agents.

Performance status is typically mentioned in the unstructured free text, particularly progress notes. As such, various metrics mentioned such as Eastern Cooperative Oncology Group (ECOG), Karnofsky Performance Status Scale (KPS), Lansky Performance Status Scale, and Palliative Performance Scale (PPS) are extracted. These different metrics are mapped to ECOG for ease of comparison. This is extracted using an information extraction module similar to the PD-L1 extraction pipeline described above. All the extracted metrics are converted to ECOG.

Smoking history or status for patients was extracted from available data about patient diagnosis, represented by ICD-10 codes. The specificity of using ICD-9 codes was high, “indicating the exceptional utility of these codes for identifying true smokers” and supporting “the use of these codes for the identification of smokers for clinical studies.” However, using NLP on clinical notes combined with the ICD-9 codes resulted in higher sensitivity. These ICD-9 codes (e.g., 305.1 Tobacco use disorder, and V15.82 Personal history of tobacco use) were converted to ICD-10, by including more specific codes that represent “tobacco”, “smoking,” “nicotine,” “cigarettes” use/abuse while filtering out instances of explicitly non-smoking use like “chewing tobacco.” Other codes that indicate use or abuse of tobacco currently or in the past, in addition to chronic exposure of environmental smoke were also included.

Patients with central nervous system (CNS) metastasis, also referred to as brain metastasis in the dataset, were collected by filtering for ICD-10 code C79.31 (secondary malignant neoplasm of brain) from patient history, the code for secondary brain or spinal cord neoplasms, 198.3 (converts approximately to ICD-10-CM C79.31), had good recall (sensitivity), precision (positive predictive value), and specificity for identifying patients with brain metastases. The precision and specificity increased when the code recurred on different days.

Lab test results are found in large amounts in structured form (between 69.3% to 76.9%) and thus we have not extracted this from the unstructured text, even though it is also commonly mentioned there. Commonly encountered lab test names were filtered for each lab test type from our dataset and each test was categorized into one of three standard units: upper limits of normal (ULN), grams per deciliter (g/dL), and count per microliter (/muL). The values are then converted to the required units. To calculate ULN, the value of the lab is divided by the higher limit of normal reference range. Each lab test had between 2.7 million to 4.99 million entries in the final dataset. Other fluid measurement units were also converted to g/dL or count/microliter. Blood pressure was found primarily in unstructured free text form but was not used in these examples.

In examples, a hybrid approach to deal with missing data was used. This is due to the structure of the missingness and their numbers across the dataset. In the structured dataset there are missing values in the line of therapy, start, diagnosis and end date and in all of the lab tests. The missing values in the laboratory tests variables (ALT, AST, hemoglobin, etc.), smoking and CNS metastasis are imputed using an in-house implementation. The line of therapy is missing in 90% of the patients. The system 100 filters it for each trial for the patients in which it is available, and it is assumed as right for those in which is missing. When there is missing data in the dates, or the combinations of dates result in an incoherent number, that patient is removed from the dataset.

In examples, the missingness of the categorical variables is addressed via a one-hot encoding. All the data included in the trials in this study were imputed simultaneously. The dimension of the latent variable was fixed to six. The width of the multi-layer perceptron in the variational auto-encoder was fixed to 32 and the decoder used a depth of 3 layers with ReLU activation functions. The training was carried out for 500 epochs and 100 samples were used for estimating the distribution of the unobserved data given the observed data. The imputation of the missing data was done as described in Mattei and Frellsen (2019).

In examples, survival outcomes are defined as (Yᵢ, Dᵢ). Dᵢ∈{0,1} represents the death event based on the follow up of the patient. Yᵢ∈Z⁺ represents the observed survival time, in days, since the start of the therapy. Yᵢ is computed as Yᵢ=min(Tᵢ, Cᵢ) where Tᵢ is the time of death and Cᵢ is the censoring time. To make the simulation of the trials as accurate as possible, the duration of the trial is used as censoring time for those patients with a larger duration. When the event death is observed, Yᵢ accounts for the number of days between the start of therapy and the event. When death is not observed, the last contact date with the patient is used and considered censored. The treatment that the patient receives is denoted by Wᵢ∈{0,1}. This is a binary variable because the system 100 only considers two-arm trials in this example, but this can be generalized further.

In examples, the covariates of the model are described by Xᵢ and include age, gender smoking, histology, CNS metastasis, ECOG score, race and the days between diagnostics and treatment. Because patients may be exposed to multiple drugs, the system 100 also keeps a record of all the drugs that each patient took at least once beyond the drug of interest. For each patient, this results in a high-dimensional binary vector Zᵢ that accounts for all the drugs that the patient took with respect to a baseline dictionary of available drugs. Because the distribution of drugs has a very long tail, in each trial the drugs that were taken by at least 20% of the patients.

In examples, simulating a trial uses some assumptions in these examples. The standard potential outcomes point of view is followed. Further, the following assumptions were used, in some examples:
1. Conditional exchangeability, Y(W=w)⊥W|X, for a=0, 1, where Y(W=w) is the potential outcome of an individual assigned to group W=w. This assumption specifies that the right confounders have been observed, which implies that conditioning on X the outcome of the treatment of an individual once it has received a treatment is independent of the selection mechanism (a randomized experiment can be simulated.
2. Positive support, P(X)>0. All eligible individuals can be selected for the study.
3. Overlap, 1>P (W=1|X)>0. All the eligible individuals have a non-null probability of being selected as cases and controls.
4. Stable Unit Treatment Value Assumption (SUTVA). There is no interference between patients. The SUTVA assumption is actually two assumptions rolled into one. First, it states that the potential outcomes for any patient do not vary with the treatments assigned to other units (patients are independent in their responses). Second, for each unit, there are no different forms or versions of each treatment level that lead to different potential outcomes (the treatment is always the same). Note that SUTVA does not say anything about how the treatment assigned to one unit affects the treatment assigned to another unit.
5. Trial population match, P_{RCT}(X)=P_{RWD}(X). When the goal is to simulate the result of a previous trial, the distribution of the confounders in the observational study and in the trial should be the same.

Assumptions 2-5 can be tested from observation data. Assumption 1 cannot, but indications can be found in the data that the confounder in the data are removing the right biases.

To evaluate the balancing between treatments and control, the propensity score (PS) is computed. The PS of a patient i is defined as:

$$e ( {X}_{i} ) = ℙ ( {W}_{i} \vert {X}_{i} ) .$$

It captures the probability taking the treatment Wᵢ in the presence of the confounders Xᵢ. In balanced randomized experiments it holds that e(Xᵢ)=0.5 for all patients. The propensity score is a balancing score. This means that by conditioning on the propensity score it is expected for the distribution of observed covariates to be the same in the treatment and the control groups. Propensity scores are used to reduce the bias due to confounding in observational studies by re-weighting the observations in the model of the outcomes. The weights of individuals with a propensity score close to 1 or 0 is reduced. These are individuals whose assignment is identified to be heavily affected by the presence of the values of the confounding variables. On the other hand, individuals with propensity score close to 0.5 are over-weighted because their assignment is independent of the covariates, as it is the case in RCTs where individuals are assigned randomly to cases and controls. In the codebase of the system 100, the propensity scores are computed with a logistic regression (e.g., LogisticRegression class in scikit-learn9).

The hazard rate of an individual, denoted usually by h(t|X, W) is the probability that a patient will die at time, t, in the presence of the covariates X and treatment W. In survival analysis, the Hazard Ratio for the treatment is defined as:

$$H R ( t ) = \frac{h ( t \vert X , W = 1 )}{h ( t \vert X , W = 0 )} ,$$

which accounts for the relative risk of death at time t between the treatment and control groups.

In examples, the system 100 uses the Cox Proportional Hazards model (Cox-PH) to perform the survival analysis. This model assumes that:

$$h ( t \vert X ) = {h}_{0} ( t ) exp ( {b}_{w} W + {\sum }_{j = 1}^{p} {b}_{j} {X}_{j} ) ,$$

where h₀(t) is known as the baseline-hazard, b_{w} is the parameter accounting for the effects of the treatment, and bⱼ is accounting for the effect of the jth covariate. The particular factorization of Cox-PH allows to associate each parameter of the model with the HR of each covariate. In particular, the HR for the treatment is:

$$H R = exp ( {b}_{w} ) ,$$

which is independent of the time due to the structure of the CoxPH model. In these examples, the class CoxPHFitter from the library lifelines is used. Two different approaches are used to compute the Hazard Ratio:
Unadjusted Cox proportional hazard (CoxPH-U): This model is used as a baseline. No confounding correction is used. The target treatment W and a vector the vector of extra drugs Z as covariates. The reported HR is the exponential of the target treatment parameter; and
Cox proportional hazard with inverse propensity re-weighting (CoxPH-IPSW): the Hazard ratio is computed using a CoxPH model trained on W and Z using an inverse propensity treatment weighting with stabilization. The weights for each patient are obtained as:

$${w}_{i} = {W}_{i} + ( 1 - {W}_{i} ) [ \frac{e ( {X}_{i} )}{1 - e ( {X}_{i} )} ] .$$

In examples, for each trial, a series of tests were performed to guarantee the stability and coherence of the results. Causal assumptions like the conditional exchangeability were not tested with observational data only. However, a sensitivity analysis of the HR with respect to several varying aspects in the data can help to identify potential issues. This study performed the following analysis:
Addition of random common cause (Sensitivity-RCC): A simulated confounder was added to the problem by randomly generating data from a Gaussian variable with mean zero and variance θ. The HR was computed for a grid of 10 values on θ∈[0.1, 5]. Each experiment is repeated 300 times. Expected positive test result: No statistical variation of the Hazard Ratio across different values of θ. This test aims to test the stability of the chosen confounders;
Placebo treatment (Sensitivity-PT): The assignment was randomly permuted to the treatment. The experiment was repeated 300 times. Expected positive test result: The newly computed HRs are not statistically significant (HR=1). This test aims to validate that the quality of the signal;
Subsets of different size (Sensitivity-SubSets). Samples of decreasing sizes are selected with replacement and HRs are recomputed. Subsamples of size 90, 75, 50 and 25% of the original dataset are taken. Each experiment is repeated 30 times. Expected positive test result: Same average HR in each scenario with an increase in variance when the sample size decreases. This test aims to capture the stability of the results and its consistence under random variations in the sample.
   Beyond indications for potentially missing confounders, it is important to evaluate that given the chosen confounders the balancing between the groups has been carried out correctly and that the structure of the data allows for causal analysis. To evaluate these issues, the following tests are performed in some examples:
Covariate balancing (Cov-Balancing): To test for the quality of the signal, and how imbalances between the groups are corrected when using the IPSW approach, the standard mean difference is computed for all the confounders with and without re-weighting for the treatment and control groups. Expected positive test result: Re-weighting makes the differences between the treatment and control groups close to zero in all confounding variables;
Positivity and overlap (Boolean set). Test that all patients have a non-negative probability of being assigned to either of the two groups (e.g., using the method described in Oberst et al. (2020) and the available in “dowhy” (Sharma and Kiciman, 2020). Expected positive test result: A large percentage of the same (>95%) satisfies the condition. This test aims to capture the overlap of cases and controls (the more overlap we have the better the data are for causal analysis).

The HR is a quantity that describes a population of individuals characterized by P(X). Therefore, for the HRs in the RCT and the observational to be comparable it is needed that P_{RCT}(X)=P_{RWE}(X). Filtering patients in the observational dataset according to the eligibility criteria helps ensure that the support of these two densities are the same so further validation is needed. Unfortunately, data from the RCT are rarely available. To test these assumptions we use the summary statistics that we published together with the RCTs and we compare those with the equivalent values in the structured EHRs.

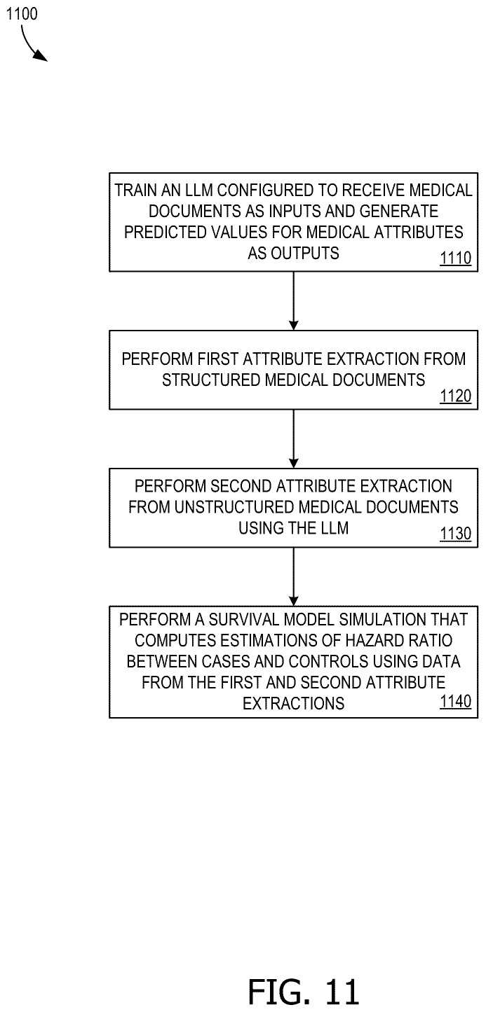

FIG. 11 is a flowchart 1100 illustrating exemplary operations that may be performed by the system 100 of FIG. 1 for performing trial simulations using patient medical data extracted from structured data sources and unstructured/semi-structured data sources. In some examples, the operations of flowchart 1100 may be similar to the operations shown and described in FIG. 1, FIG. 2, and/or FIG. 4. In the example implementation, the operations of flowchart 1100 are performed by the trial simulation device 110 of FIG. 1.

At operation 1110, the trial simulation device 110 trains a large language model (LLM) (e.g., LLM 122 of FIG. 1). The LLM is configured to receive, as at least one input, a medical document that includes medical text associated with a patient and generate, as at least one output in response to the at least one input, one or more predicted values for one or more medical attributes of the patient based on the medical text included in the medical document. In some examples, training the LLM further comprises training the LLM using a plurality of labeled training documents, each labeled training document being labeled with a value for at least one labeled attribute.

At operation 1120, the trial simulation device 110 performs first attribute extraction from a plurality of structured medical documents of a plurality of patients (e.g., structured data docs 212 of FIG. 2), thereby extracting values for a first plurality of attributes associated with the plurality of patients (e.g., structured attributes 216 of FIG. 2). At operation 1130, the trial simulation device 110 performs second attribute extraction from a plurality of unstructured medical documents of the plurality of patients (e.g., unstructured/semi-structured data docs 214) using the LLM, thereby extracting predicted values for a second plurality of attributes associated with the plurality of patients (e.g., predicted attributes 218 of FIG. 2). In some examples, performing the first attribute extraction further comprises storing the values for the first plurality of attributes in a matrix (e.g., matrix 220 of FIG. 2) that identifies unique patients in a first dimension (e.g., as patients 222) of the matrix and unique attributes in a second dimension (e.g., as attributes 224) of the matrix, wherein each cell in the matrix stores one value of an associated attribute for a particular patient, wherein performing the second attribute extraction further comprises storing the predicted values for the second plurality of attributes associated with the plurality of patients in the matrix.

At operation 1140, the trial simulation device 110 performs a survival model simulation that computes estimations of hazard ratio (HR) between cases and controls using real-world data of the plurality of patients extracted in the first attribute extraction and second attribute extraction.

In some examples, the trial simulation device 110 also generates second predicted values for one or more attributes of the second plurality of attributes using a latent variable model (e.g., latent variable model 234 of FIG. 2) and updates values of the one or more attributes with the second predicted values.

In some examples, the LLM is further configured to generate a confidence score for each value of the one or more predicted values, wherein generating the second predicted values further comprises generating second predicted values for one or more attributes of the second plurality of attributes when an associated confidence score is below a threshold.

In some examples, the trial simulation device 110 is further configured to identify a plurality of eligible patients (e.g., eligible patients 244 of FIG. 2) from the matrix based on eligibility criteria (e.g., eligibility criteria 152 of FIG. 1 and FIG. 2) and create a trial matrix (e.g., trial matrix 250 of FIG. 2) that includes data associated with the plurality of eligible patients from the matrix, and wherein performing the survival model simulation includes using the trial matrix as input data for the survival model simulation.

In some examples, the trial simulation device 110 is further configured to perform a test diagnostic on output of the survival model simulation to evaluate a quality of the survival model simulation.

### Additional Examples

An example trial simulation system comprises: at least one processor; a large language model (LLM) configured to: receive, as at least one input, a medical document that includes medical text associated with a patient; and generate, as at least one output in response to the at least one input, one or more predicted values for one or more medical attributes of the patient based on the medical text included in the medical document; an attribute extraction module configured to: perform first attribute extraction from a plurality of structured medical documents of a plurality of patients, thereby extracting values for a first plurality of attributes associated with the plurality of patients; and perform second attribute extraction from a plurality of unstructured medical documents of the plurality of patients using the LLM, thereby extracting predicted values for a second plurality of attributes associated with the plurality of patients; and a trial simulation module configured to perform a survival model simulation that computes estimations of hazard ratio (HR) between cases and controls using real-world data of the plurality of patients extracted in the first attribute extraction and second attribute extraction.

An example computer-implemented method comprises: training a large language model (LLM), the LLM being configured to: receive, as at least one input, a medical document that includes medical text associated with a patient; and generate, as at least one output in response to the at least one input, one or more predicted values for one or more medical attributes of the patient based on the medical text included in the medical document; performing first attribute extraction from a plurality of structured medical documents of a plurality of patients, thereby extracting values for a first plurality of attributes associated with the plurality of patients; performing second attribute extraction from a plurality of unstructured medical documents of the plurality of patients using the LLM, thereby extracting predicted values for a second plurality of attributes associated with the plurality of patients; and performing a survival model simulation that computes estimations of hazard ratio (HR) between cases and controls using real-world data of the plurality of patients extracted in the first attribute extraction and second attribute extraction.

An example computer storage device has computer-executable instructions stored thereon, which, on execution by a computer, cause the computer to perform operations comprising: training a large language model (LLM), the LLM being configured to: receive, as at least one input, a medical document that includes medical text associated with a patient; and generate, as at least one output in response to the at least one input, one or more predicted values for one or more medical attributes of the patient based on the medical text included in the medical document; performing first attribute extraction from a plurality of structured medical documents of a plurality of patients, thereby extracting values for a first plurality of attributes associated with the plurality of patients; performing second attribute extraction from a plurality of unstructured medical documents of the plurality of patients using the LLM, thereby extracting predicted values for a second plurality of attributes associated with the plurality of patients; and performing a survival model simulation that computes estimations of hazard ratio (HR) between cases and controls using real-world data of the plurality of patients extracted in the first attribute extraction and second attribute extraction.

Alternatively, or in addition to the other examples described herein, examples include any combination of the following:
training a large language model (LLM) that is configured to receive, as at least one input, a medical document that includes medical text associated with a patient;
training an LLM that is configured to generate, as at least one output, one or more predicted values for one or more medical attributes of the patient based on medical text included an input document;
performing first attribute extraction from a plurality of structured medical documents of a plurality of patients, thereby extracting values for a first plurality of attributes associated with the plurality of patients;
performing second attribute extraction from a plurality of unstructured medical documents of the plurality of patients using the LLM, thereby extracting predicted values for a second plurality of attributes associated with the plurality of patients; and
performing a survival model simulation that computes estimations of hazard ratio (HR) between cases and controls using real-world data of the plurality of patients extracted in the first attribute extraction and second attribute extraction;
training the LLM using a plurality of labeled training documents, each labeled training document being labeled with a value for at least one labeled attribute;
generating second predicted values for one or more attributes of the second plurality of attributes using a latent variable model;
updating values of the one or more attributes with the second predicted values;
training an LLM that is configured to generate a confidence score for each value of the one or more predicted values;
generating second predicted values for one or more attributes of the second plurality of attributes when an associated confidence score is below a threshold;
a matrix that identifies unique patients in a first dimension of the matrix and unique attributes in a second dimension of the matrix;
performing the first attribute extraction further comprises storing the values for the first plurality of attributes in a matrix that identifies unique patients in a first dimension of the matrix and unique attributes in a second dimension of the matrix;
each cell in a matrix stores one value of an associated attribute for a particular patient;
storing predicted values for the second plurality of attributes associated with the plurality of patients in the matrix;
identifying a plurality of eligible patients from the matrix based on eligibility criteria;
creating a trial matrix that includes data associated with the plurality of eligible patients from the matrix;
using the trial matrix as input data for the survival model simulation; and
performing a test diagnostic on output of the survival model simulation to evaluate a quality of the survival model simulation.

While the aspects of the disclosure have been described in terms of various examples with their associated operations, a person skilled in the art would appreciate that a combination of operations from any number of different examples is also within scope of the aspects of the disclosure.

### Example Operating Environment

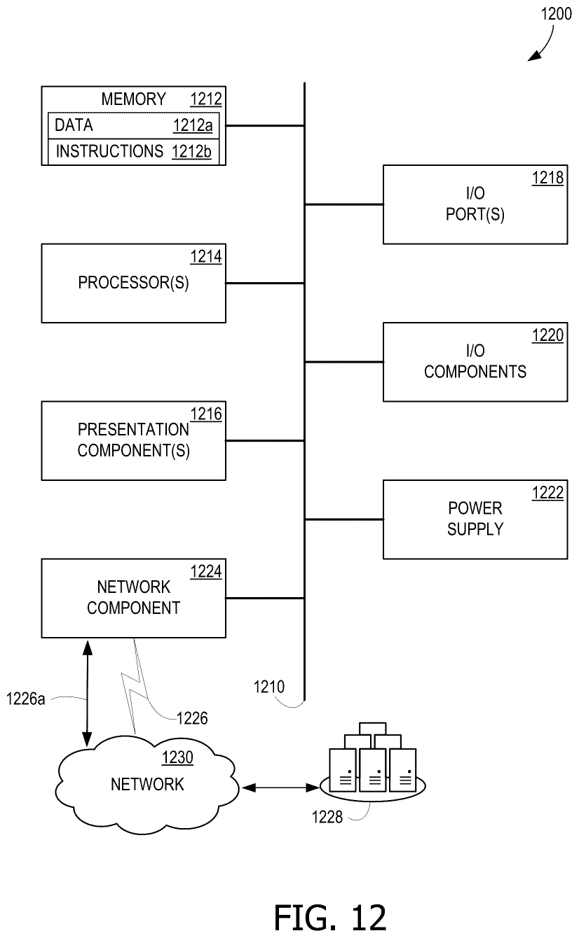

FIG. 12 is a block diagram of an example computing device 1200 (e.g., a computer storage device) for implementing aspects disclosed herein and is designated generally as computing device 1200. In some examples, one or more computing devices 1200 are provided for an on-premises computing solution. In some examples, one or more computing devices 1200 are provided as a cloud computing solution. In some examples, a combination of on-premises and cloud computing solutions are used. Computing device 1200 is but one example of a suitable computing environment and is not intended to suggest any limitation as to the scope of use or functionality of the examples disclosed herein, whether used singly or as part of a larger set. Neither should computing device 1200 be interpreted as having any dependency or requirement relating to any one or combination of components/modules illustrated.

The examples disclosed herein may be described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments when tasks are performed by remote-processing devices that are linked through a communications network.

Computing device 1200 includes a bus 1210 that directly or indirectly couples the following devices: computer storage memory 1212, one or more processors 1214, one or more presentation components 1216, input/output (I/O) ports 1218, I/O components 1220, a power supply 1222, and a network component 1224. While computing device 1200 is depicted as a seemingly single device, multiple computing devices 1200 may work together and share the depicted device resources. For example, memory 1212 may be distributed across multiple devices, and processor(s) 1214 may be housed with different devices.

Bus 1210 represents what may be one or more busses (such as an address bus, data bus, or a combination thereof). Although the various blocks of FIG. 6 are shown with lines for the sake of clarity, delineating various components may be accomplished with alternative representations. For example, a presentation component such as a display device is an I/O component in some examples, and some examples of processors have their own memory. Distinction is not made between such categories as “workstation,” “server,” “laptop,” “hand-held device,” etc., as all are contemplated within the scope of FIG. 6 and the references herein to a “computing device.” Memory 1212 may take the form of the computer storage media referenced below and operatively provide storage of computer-readable instructions, data structures, program modules and other data for the computing device 1200. In some examples, memory 1212 stores one or more of an operating system, a universal application platform, or other program modules and program data. Memory 1212 is thus able to store and access data 1212a and instructions 1212b that are executable by processor 1214 and configured to carry out the various operations disclosed herein.

In some examples, memory 1212 includes computer storage media. Memory 1212 may include any quantity of memory associated with or accessible by the computing device 1200. Memory 1212 may be internal to the computing device 1200 (as shown in FIG. 6), external to the computing device 1200 (not shown), or both (not shown). Additionally, or alternatively, the memory 1212 may be distributed across multiple computing devices 1200, for example, in a virtualized environment in which instruction processing is carried out on multiple computing devices 1200. For the purposes of this disclosure, “computer storage media,” “computer-storage memory,” “memory,” and “memory devices” are synonymous terms for the computer-storage memory 1212, and none of these terms include carrier waves or propagating signaling.

Processor(s) 1214 may include any quantity of processing units that read data from various entities, such as memory 1212 or I/O components 1220. Specifically, processor(s) 1214 are programmed to execute computer-executable instructions for implementing aspects of the disclosure. The instructions may be performed by the processor, by multiple processors within the computing device 1200, or by a processor external to the client computing device 1200. In some examples, the processor(s) 1214 are programmed to execute instructions such as those illustrated in the flow charts discussed below and depicted in the accompanying drawings. Moreover, in some examples, the processor(s) 1214 represent an implementation of analog techniques to perform the operations described herein. For example, the operations may be performed by an analog client computing device 1200 and/or a digital client computing device 1200. Presentation component(s) 1216 present data indications to a user or other device. Exemplary presentation components include a display device, speaker, printing component, vibrating component, etc. One skilled in the art will understand and appreciate that computer data may be presented in a number of ways, such as visually in a graphical user interface (GUI), audibly through speakers, wirelessly between computing devices 1200, across a wired connection, or in other ways. I/O ports 1218 allow computing device 1200 to be logically coupled to other devices including I/O components 1220, some of which may be built in. Example I/O components 1220 include, for example but without limitation, a microphone, joystick, game pad, satellite dish, scanner, printer, wireless device, etc.

Computing device 1200 may operate in a networked environment via the network component 1224 using logical connections to one or more remote computers. In some examples, the network component 1224 includes a network interface card and/or computer-executable instructions (e.g., a driver) for operating the network interface card. Communication between the computing device 1200 and other devices may occur using any protocol or mechanism over any wired or wireless connection. In some examples, network component 1224 is operable to communicate data over public, private, or hybrid (public and private) using a transfer protocol, between devices wirelessly using short range communication technologies (e.g., near-field communication (NFC), Bluetooth™ branded communications, or the like), or a combination thereof. Network component 1224 communicates over wireless communication link 1226 and/or a wired communication link 1226a to a remote resource 1228 (e.g., a cloud resource) across network 1230. Various different examples of communication links 1226 and 1226a include a wireless connection, a wired connection, and/or a dedicated link, and in some examples, at least a portion is routed through the internet.

Although described in connection with an example computing device 1200, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals. Exemplary computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A trial simulation system comprising:
a processor;
a large language model (LLM) configured to:
receive, as at least one input, a medical document that includes medical text associated with a patient; and
generate, as at least one output in response to the at least one input, one or more predicted values for one or more medical attributes of the patient based on the medical text included in the medical document;
an attribute extraction module configured to:
perform first attribute extraction from a plurality of structured medical documents of a plurality of patients, including extracting values for a first plurality of attributes associated with the plurality of patients; and
perform second attribute extraction from a plurality of unstructured medical documents of the plurality of patients using the LLM, including extracting predicted values for a second plurality of attributes associated with the plurality of patients; and
a trial simulation module configured to perform a survival model simulation that computes estimations of hazard ratio (HR) between cases and controls using real-world data of the plurality of patients extracted in the first attribute extraction and second attribute extraction.

2. The trial simulation system of claim 1, further comprising an LLM training module configured to train the LLM using a plurality of labeled training documents, each labeled training document being labeled with a value for at least one labeled attribute.

3. The trial simulation system of claim 1, further comprising a latent variable module configured to:
generate second predicted values for one or more attributes of the second plurality of attributes using a latent variable model; and
update values of the one or more attributes with the second predicted values.

4. The trial simulation system of claim 3, wherein the LLM is further configured to generate a confidence score for each value of the one or more predicted values, wherein generating the second predicted values further comprises generating second predicted values for one or more attributes of the second plurality of attributes when an associated confidence score is below a threshold.

5. The trial simulation system of claim 1, wherein performing the first attribute extraction further comprises storing the values for the first plurality of attributes in a matrix that identifies unique patients in a first dimension of the matrix and unique attributes in a second dimension of the matrix, wherein each cell in the matrix stores one value of an associated attribute for a particular patient, wherein performing the second attribute extraction further comprises storing the predicted values for the second plurality of attributes associated with the plurality of patients in the matrix.

6. The trial simulation system of claim 5, further comprising a patient selection module configured to:
identify a plurality of eligible patients from the matrix based on eligibility criteria; and
create a trial matrix that includes data associated with the plurality of eligible patients from the matrix,
wherein performing the survival model simulation includes using the trial matrix as input data for the survival model simulation.

7. The trial simulation system of claim 1, further comprising an analytics module configured to perform a test diagnostic on output of the survival model simulation to evaluate a quality of the survival model simulation.

8. A computer-implemented method comprising:
training a large language model (LLM), the LLM being configured to:
receive, as at least one input, a medical document that includes medical text associated with a patient; and
generate, as at least one output in response to the at least one input, one or more predicted values for one or more medical attributes of the patient based on the medical text included in the medical document;
performing first attribute extraction from a plurality of structured medical documents of a plurality of patients, including extracting values for a first plurality of attributes associated with the plurality of patients;
performing second attribute extraction from a plurality of unstructured medical documents of the plurality of patients using the LLM, including extracting predicted values for a second plurality of attributes associated with the plurality of patients; and
performing a survival model simulation that computes estimations of hazard ratio (HR) between cases and controls using real-world data of the plurality of patients extracted in the first attribute extraction and second attribute extraction.

9. The method of claim 8, wherein training the LLM further comprises training the LLM using a plurality of labeled training documents, each labeled training document being labeled with a value for at least one labeled attribute.

10. The method of claim 8, further comprising:
generating second predicted values for one or more attributes of the second plurality of attributes using a latent variable model; and
updating values of the one or more attributes with the second predicted values.

11. The method of claim 10, wherein the LLM is further configured to generate a confidence score for each value of the one or more predicted values, wherein generating the second predicted values further comprises generating second predicted values for one or more attributes of the second plurality of attributes when an associated confidence score is below a threshold.

12. The method of claim 8, wherein performing the first attribute extraction further comprises storing the values for the first plurality of attributes in a matrix that identifies unique patients in a first dimension of the matrix and unique attributes in a second dimension of the matrix, wherein each cell in the matrix stores one value of an associated attribute for a particular patient, wherein performing the second attribute extraction further comprises storing the predicted values for the second plurality of attributes associated with the plurality of patients in the matrix.

13. The method of claim 12, further comprising:
identifying a plurality of eligible patients from the matrix based on eligibility criteria; and
creating a trial matrix that includes data associated with the plurality of eligible patients from the matrix,
wherein performing the survival model simulation includes using the trial matrix as input data for the survival model simulation.

14. The method of claim 8, further comprising performing a test diagnostic on output of the survival model simulation to evaluate a quality of the survival model simulation.

15. A computer storage device having computer-executable instructions stored thereon, which, on execution by a computer, cause the computer to perform operations comprising:
training a large language model (LLM), the LLM being configured to:
receive, as at least one input, a medical document that includes medical text associated with a patient; and
generate, as at least one output in response to the at least one input, one or more predicted values for one or more medical attributes of the patient based on the medical text included in the medical document;
performing first attribute extraction from a plurality of structured medical documents of a plurality of patients, including extracting values for a first plurality of attributes associated with the plurality of patients;
performing second attribute extraction from a plurality of unstructured medical documents of the plurality of patients using the LLM, including extracting predicted values for a second plurality of attributes associated with the plurality of patients; and
performing a survival model simulation that computes estimations of hazard ratio (HR) between cases and controls using real-world data of the plurality of patients extracted in the first attribute extraction and second attribute extraction.

16. The computer storage device of claim 15, wherein training the LLM further comprises training the LLM using a plurality of labeled training documents, each labeled training document being labeled with a value for at least one labeled attribute.

17. The computer storage device of claim 15, the operations further comprising:
generating second predicted values for one or more attributes of the second plurality of attributes using a latent variable model; and
updating values of the one or more attributes with the second predicted values.

18. The computer storage device of claim 17, wherein the LLM is further configured to generate a confidence score for each value of the one or more predicted values, wherein generating the second predicted values further comprises generating second predicted values for one or more attributes of the second plurality of attributes when an associated confidence score is below a threshold.

19. The computer storage device of claim 15, wherein performing the first attribute extraction further comprises storing the values for the first plurality of attributes in a matrix that identifies unique patients in a first dimension of the matrix and unique attributes in a second dimension of the matrix, wherein each cell in the matrix stores one value of an associated attribute for a particular patient, wherein performing the second attribute extraction further comprises storing the predicted values for the second plurality of attributes associated with the plurality of patients in the matrix.

20. The computer storage device of claim 19, the operations further comprising:
identifying a plurality of eligible patients from the matrix based on eligibility criteria; and
creating a trial matrix that includes data associated with the plurality of eligible patients from the matrix,
wherein performing the survival model simulation includes using the trial matrix as input data for the survival model simulation.
